(19) United States
(12) Patent Application Publication
(10) Pub. No.: US 2007/0220621 A1
(43) Pub. Date: Sep. 20, 2007

(54) Genetic characterization and prognostic significance of cancer stem cells in cancer

(76) Inventors: Michael F. Clarke, Palo Alto, CA (US); Xinhao Wang, Fremont, CA (US); Robert W. Cho, San Jose, CA (US)

Correspondence Address: MEDLEN & CARROLL, LLP, Suite 350, 101 Howard Street, San Francisco, CA, 94105, US

(21) Appl. No.: 11/591,122

(22) Filed: Oct. 31, 2006

Related U.S. Application Data
(60) Provisional application No. 60/731,470, filed on Oct. 31, 2005.

(51) Int. Cl. A01K 67/027; C12Q 1/68; G01N 33/574; C12N 5/08

(52) U.S. Cl. 800/18; 435/6; 435/325; 435/366; 435/7.23

(57) ABSTRACT

The present invention is related to the identification of cancer stem cells using the MMTV-Wnt-1 transgenic mouse model. These cancer stem cells have a gene expression signature that allows them to be distinguished from their non-tumorigenic counterparts. Moreover, the gene expression pattern can also predict survival in a diverse group of solid tumors.

### CROSS REFERENCE TO RELATED APPLICATIONS

This application claims the benefit of U.S. Appl. No. 60/731,470, filed Oct. 31, 2005, which is herein incorporated by reference in its entirety.

### STATEMENT REGARDING FEDERALLY-SPONSORED RESEARCH AND DEVELOPMENT

This invention was made with government support under CA104987 awarded by the National Institute of Health. The U.S. Government has certain rights in this invention.

### FIELD OF THE INVENTION

The present invention is related to the identification of murine breast cancer stem cells using the MMTV-Wnt-1 transgenic mouse model. These cells have a gene expression signature which allows them to be distinguished from their non-tumorigenic counterparts. Moreover, the gene expression pattern can also predict survival in a diverse group of solid tumors.

### BACKGROUND

A new model of tumorigenesis has emerged in which many types of malignant tumors contain a minority population of cells akin to stem cells. In this model, a tumor is composed of a heterogeneous population of cells that are all derived from a small sub-population of cancer cells that have the exclusive ability to self-renew and extensively proliferate and differentiate, while the rest of the cancer cells lack this ability (Reya, T. et al., Nature 414:105-11 (2001)). In acute myeloid leukemia (AML), only a subpopulation of cells are capable of driving leukemic engraftment in NOD/SCID mice (Bonnet, D. and Dick, J. Nat. Med. 3:730-737 (1997); Lapidot, T. et al. Nature 17:645-648 (1994)). These leukemia initiating cells (L-ICs) share some of the same cell surface markers as normal hematopoietic stem cells, leading to the suggestion that the L-ICs may have arisen from a normal hematopoietic stem cell whose normal capacity for self-renewal is deregulated. In patients with chronic myelogenous leukemia (CML) in blast crisis, the granulocyte progenitor cell has been identified as the cells from which L-ICs arise in this disease, and therefore, leukemogenesis likely occurs as a result of a committed progenitor cell that has acquired the ability to self-renew (Jamieson, C. H. et al., N Engl J Med 351:657-67 (2004)). Thus, cancer stem cells may be derived from normal stem cells with dysregulated self-renewal pathways, or from a more differentiated, progenitor cell type that has acquired the ability to self-renew.

Recently, a small sub-population of cancer cells that have the exclusive ability to regenerate tumors has been prospectively identified in some human solid tumors. It has been demonstrated that a small population of tumorigenic cells, sometimes referred to as cancer stem cells (CSCs), can be prospectively isolated from human breast tumors. These cells could be identified by the expression of the cell surface markers CD44⁺CD24^{-/lo}Lineage⁻, and were capable of regenerating the phenotypic heterogeneity of the original tumor when injected subcutaneously into NOD/SCID mice (Al-Hajj, M. et al., Proc. Natl. Acad. Sci. USA 100:3983-8 (2003)). In the tumors of most patients, these cells represent a minority population of cancer cells and are capable of being serially passaged and retain their ability to reconstitute the heterogeneic population of cells within the original tumor with serial passages, using as few as 100 cells in an injection. The remaining bulk of cancer cells that are not CD44⁺CD24^{-/lo} are unable to form tumors. Similarly, a tumorigenic cell population has been isolated from human brain tumors, characterized by the expression of CD133 (Singh, G. Nature (2004)).

In normal stem cells of adult tissues, the process of self-renewal is tightly regulated as to prevent an uncontrolled expansion of the stem cell pool. Disruption of genes involved in this regulation likely results in unlimited expansion of self-renewing cells and may form the basis of tumorigenesis. Predictably, it has been shown that some oncogenes function to regulate self-renewal, including the Wnt/β-catenin signaling pathway, which plays a pivotal role in both self-renewal of normal stem cells and malignant transformation (Cadigan, K. M. and Nusse, R. Genes & Development 11:3286-305 (1997); Spink, K. E. et al., Embo J 19:2270-9 (2000); Austin, T. et al., Blood 89:3624-35 (1997)).

The Wnt pathway was first discovered in mouse mammary tumor virus (MMTV) induced murine breast tumors in which proviral insertion resulted in deregulated expression of Wnt-1, promoting the formation of mammary tumors (Nusse, R. et al. Nature 307:131-6 (1984)). Wnt signaling leads to the stabilization and accumulation of β-catenin which is normally targeted for degradation. Consequently, β-catenin, after translocation into the nucleus, binds to the LEF/TCF family of transcription factors resulting in the activated transcription of genes that promote proliferation. Mutations in the Wnt signaling pathway that result in the constitutive activation of β-catenin have been implicated as the mechanism of tumorigenesis in colon as well as in certain brain and skin tumors (van de Wetering, M. et al. Cell 111:241-50 (2002)). Although breast cancer mutations in APC or β-catenin are rare (Schlosshauer, P. W. et al. Carcinogenesis 21:1453-6 (2000), recent studies suggest that autocrine Wnt secretion activates the canonical (β-catenin signaling pathway in approximately 25% of breast cancer cell lines that were tested (Bafico, A., et al. Cancer Cell 6:497-506 (2004)).

### BRIEF SUMMARY OF THE INVENTION

A new population of murine breast cancer stem cells have been discovered using the MMTV-Wnt-1 transgenic mouse model. These cells have a gene expression signature which allows them to be distinguished from their non-tumorigenic counterparts. Moreover, the gene expression pattern can also predict survival in a diverse group of solid tumors.

The invention is directed to an isolated population of murine solid tumor stem cells, the population comprising at least 75% solid tumor stem cells and less than 25% solid tumor cells, wherein the solid tumor stem cells: (i) are tumorigenic upon serial transplantation into an immunocompromised mouse; (ii) express Thy1, CD24, CD49f; and (iii) do not express detectable levels of CD45; and wherein the solid tumor cells are non-tumorigenic.

The solid tumor stem cells can be breast cancer stem cells. One or more of the solid tumor stem cells can contain a polynucleotide vector. The polynucleotide vector can be a viral vector or a plasmid. The polynucleotide vector can contain a reporter polynucleotide. The reporter polynucleotide can provide a detectable signal when active in a solid tumor stem cell. One or more of the solid tumor stem cells can further comprise a recombinant polynucleotide. The recombinant polynucleotide can be integrated into a chromosome of the solid tumor stem cell. The solid tumor stem cells can be capable of forming a new tumor upon serial transplantation into a host animal. The host animal can be an immunocompromised mouse. The isolated population can be situated in a culture medium. The solid tumor stem cells can be affixed to a substrate. The solid tumor stem cells can be treated to reduce proliferation. The solid tumor stem cells can be treated to increase proliferation.

The invention is also directed to an enriched population of murine solid tumor stem cells, the population comprising solid tumor stem cells and solid tumor cells, wherein the solid tumor stem cells: (a) are enriched at least two-fold; (b) are tumorigenic upon serial transplantation into an immunocompromised mouse; (c) express Thy1, CD24, CD49f; and (d) do not express detectable levels of CD45. The enriched population of murine solid tumor stem cells can be enriched at least 5-fold.

The invention is directed to a method of enriching for a population of murine solid tumor stem cells, the method comprising: (a) dissociating a murine solid tumor to obtain dissociated cells; (b) contacting the dissociated cells with a first reagent that binds Thy1, a second reagent that binds CD24, a third reagent that binds CD49f, and a fourth reagent that binds CD45; and (c) selecting solid tumor stem cells that bind the first, second and third reagents and do not bind to the fourth reagent. The method can further comprise isolating the selected solid tumor stem cells. The first, second, third or fourth reagent can be an antibody. The first, second, third or fourth reagent can be conjugated to a fluourochrome or magnetic particle. The selection of solid tumor stem cells can performed by flow cytometry, fluorescence activated cell sorting, panning, affinity column separation or magnetic separation. The murine solid tumor stem cells can be breast cancer stem cells. The dissociated cells can be contacted with the first, second, third, and fourth reagents concurrently. The method can further comprise: (d) introducing at least one selected cell to a culture medium that supports growth of tumor stem cells; and (e) proliferating the selected cell in the culture medium. The method can further comprise: (f) contacting the proliferated cell with a test compound; and (g) determining the effect of the test compound on the proliferated cell.

The invention is further directed to a method for analyzing an enriched population of solid tumor stem cells for a gene expression pattern, the method comprising: (a) obtaining an enriched population of solid tumor stem cells, wherein (i) the solid tumor stem cells are derived from a solid tumor; (ii) the solid tumor stem cells expresses the cell surface markers Thy1, CD24, and CD49f; (iii) the solid tumor stem cells do not express CD45; (iv) the solid tumor stem cells are tumorigenic upon serial transplantation into an immunocompromised mouse; and (v) the solid tumor stem cell population is enriched at least 2-fold relative to unfractionated tumor cells; and (b) analyzing the enriched population for a gene expression pattern. The analysis can be by a method selected from the group consisting of sequencing, high throughput screening, use of a microarray, use of analytical software for data collection and storage, use of analytical software for flexible formatting of data output, use of analytical software for statistical analysis of individual spot intensities to provide grouping and cluster analyses, and use of analytical software for linkage to external databases.

The invention is directed to a method for analyzing an enriched population of solid tumor stem cells for protein expression patterns, the method comprising: (a) obtaining an enriched population of solid tumor stem cells, wherein (i) the solid tumor stem cells are derived from a solid tumor; (ii) the solid tumor stem cells express the cell surface markers Thy1, CD24, and CD49f; (iii) the solid tumor stem cells do not express CD45; (iv) the solid tumor stem cells are tumorigenic upon serial transplantation into an immunocompromised mouse; and (v) the enriched population is enriched at least 2-fold for solid tumor stem cells relative to unfractionated tumor cells; and (b) analyzing the enriched population for a protein expression pattern. The analysis can be by a method selected from the group consisting of mass spectrometry, high throughput screening, use of a microarray, use of analytical software for data collection and storage, use of analytical software for flexible formatting of data output, use of analytical software for statistical analysis of individual spot intensities to provide grouping and cluster analyses, and use of analytical software for linkage to external databases.

The invention is also directed to a method for determining an effect of a test compound on a solid tumor stem cell, the method comprising: (a) obtaining a solid tumor stem cell, wherein; (i) the solid tumor stem cell is derived from a solid tumor; (ii) the solid tumor stem cell expresses the cell surface markers Thy1, CD24 and CD49f; (iii) the solid tumor stem cell does not express CD45; and (iv) the solid tumor stem cell is tumorigenic upon serial transplantation into an immunocompromised mouse; (b) contacting the solid tumor stem cell with the test compound; and (c) determining the response of the solid tumor stem cell to the test compound. The solid tumor stem cell can be a breast cancer cell. The solid tumor stem cell can be localized in a manner selected from the group consisting of: in a monolayer in culture, in suspension in culture, and affixed to a solid surface. The contacting can be effected at more than one concentration of the test compound being tested. The contacting can be effected using a microfluidic method. The determination of the response of the contacted cell to the test compound can comprise assaying for an effect selected from the group consisting of tumor formation, tumor growth, tumor stem cell proliferation, tumor cell survival, tumor cell cycle status, and tumor stem cell survival. The test compound can be attached to a solid surface. The test compound can be attached to a solid surface as a microarray. The test compound can be in a set of other molecules. The test compound can be in an array of other molecules. The method can further comprise (d) identifying the target in the contacted cell with which the test compound interacts.

The invention is directed to a method for determining an effect of a test compound on a solid tumor stem cell, the method comprising: (a) obtaining a solid tumor stem cell, wherein; (i) the solid tumor stem cell is derived from a solid tumor; (ii) the solid tumor stem cell expresses the cell surface markers Thy1, CD24 and CD49f, (iii) the solid tumor stem cell does not express CD45; and (iv) the solid tumor stem cell is tumorigenic upon serial transplantation into an immunocompromised mouse; (b) transplanting the obtained cell into an immunocompromised mouse; (c) administering a test compound to the immunocompromised mouse; and (d) determining the response of the transplanted solid tumor stem cell to the test compound. The solid tumor stem cell can be a breast cancer cell. The enriched population of solid tumor stem cells can be an isolated solid tumor stem cell.

The invention is directed to a method for producing a genetically modified solid tumor stem cell, the method comprising: (a) obtaining a solid tumor stem cell, wherein (i) the solid tumor stem cell is derived from a solid tumor; (ii) the solid tumor stem cell expresses the cell surface markers Thy1, CD24 and CD49f, (iii) the solid tumor stem cell does not express CD45; and (iv) the solid tumor stem cell is tumorigenic upon serial transplantation into an immunocompromised mouse; and (b) genetically modifying the obtained solid tumor stem cell. The solid tumor stem cell can be a breast cancer cell. The genetic modification can be performed in vitro. The genetic modification can be performed in vivo. The genetic modification can be introduction of a plasmid into the solid tumor stem cell. The genetic modification can be introduction of a viral vector into the solid tumor stem cell. The viral vector can be modified to express a protein that recognizes an antigen on the solid tumor stem cell. The method can further comprise: (c) examining the effect of the genetic modification on tumor formation, tumor growth, tumor cell proliferation, tumor cell survival, tumor stem cell survival, tumor stem cell proliferation, tumor cell cycle status, or tumor stem cell frequency.

The invention is directed to a method for determining a prognosis of a cancer patient, the method comprising: (a) obtaining a cell sample from the patient; (b) determining a gene signature pattern of the cell sample; (c) comparing the gene signature pattern of the cell sample to the gene signature pattern of an analogous murine tumor; and (d) evaluating whether the gene signature pattern of the cancer patient is similar to the gene signature pattern of the murine tumor. A positive prognosis results from a difference in the gene signature patterns. A negative prognosis results from similar gene signature patterns. The method in (b) can further comprise isolating solid tumor stem cells from the cell sample. The method in (b) can further comprise array amplification of gene signature targets. The determination of the expression levels comprising the gene signature can be by measuring the expression of a corresponding polypeptide. The polypeptide can be detected by immunohistochemical analysis on the cell sample using an antibody or antigen binding fragment that binds the polypeptide. The polypeptide can be detected by ELISA assay using an antibody or antigen binding fragment that binds the polypeptide. The polypeptide can be detected using an antibody array comprising an antibody or antigen binding fragment that binds the polypeptide. The gene signature can comprise genes in Table 1.

### BRIEF DESCRIPTION OF THE DRAWINGS/FIGURES

FIG. 1 shows that only a subset of the MMTV-Wnt-1 tumor cells have tumor forming capacities. (a) De novo MMTV Wnt-1 FVB/NJ tumors were harvested, digested into single cell suspensions and stained with CD45, a pan-hematopoietic marker. CD45⁻ cells were collected by flow cytometry. The CD45⁻ cells were injected into recipient female FVB/NJ mice in a limiting dilution fashion. Injections of less than 1000 cells did not produce tumors. 4 of 15, 12 of 25, and 5 of 6 injections of 2000, 5000, and 10,000 cell injections respectively, resulted in tumors. (b) CD24⁺CD45⁻ cells were injected at 1000 cell doses. CD24⁺CD45⁻ cells produced tumors in 15 out of 38 injections compared to 1 out of 30 for CD24⁻CD45⁻ cells. (c) Thy1.1⁺CD45⁻ cells were injected at 1000 cell doses resulting in 10 tumors out of 25 injection as compared to one out of 15 injections for Thy1⁻CD45⁺ cells. This data led to the combining of Thy1 and CD24 as selection markers. (d) Indicated cell populations from 6 different MMTV Wnt-1 tumors were collected. Denominators in the table represent the number of injections and the numerators represent the number of resultant tumors from the injected tumor cells. The top two data rows are a compilation of six experiments done with Wnt-1 tumors. Wnt-1 Thy1⁺CD24⁺ cells were able to form tumors even at 50 cell dose injections forming 5 tumors out of 15 injections. In contrast, Wnt-1 “Not Thy1⁺CD24⁺” cells were depleted of tumor forming cells as evidenced by only one tumor formation out of 12 injections at a cell dose of 10,000. *Some injections include CD49f along with CD24 and Thy1.1 as selection markers.

FIG. 2 shows that Wnt-1 tumor Thy1⁺CD24⁺CD49f⁺CD45⁻ cells make up 0.5-1% of the total tumor cells and are enriched for tumor forming cells. Panel (a) depicts FACS plots of the MMTV-Wnt-1 tumor in single cell suspension stained with CD24, Thy and depleted for CD45⁺ (hematopoietic cells) and DAPI⁺/7-AAD⁺ cells (dead or dying cells). The bold box indicates the selection gate of Thy1⁺CD24⁺CD45⁻ used to sort cells. (b) Thy1⁺CD24⁺CD49f⁺CD45⁻ cells make up 0.5%-1% of the total tumor cells. The remaining cells which do not fit the Thy1⁺CD24⁺CD49f⁺CD45⁻ profile, referred, to as “Not Thy1⁺CD24⁺CD45”, are shown in panel (c). The Thy1⁺CD24⁺CD45⁻ cells, when injected into recipient mice, resulted in tumor formation whereas the “Not Thy1⁺CD24⁺CD45⁻” cells were depleted of tumorigenic cells. (d) Resultant tumor from an injection of Thy1⁺CD24⁺CD45⁻ cells showed the same marker profile as the original tumor.

FIG. 3 shows that Wnt-1 tumor Thy1⁺CD24⁺CD45⁻ cells isolated from unpassaged and passaged tumors both have similar tumor-forming potentials. Injections of Thy1⁺CD24⁺CD45⁻ cells collected from de novo tumors are shown in panel (a). Thy1⁺CD24⁺CD45⁻ cells from de novo tumors resulted in tumor. formation in all 10 injections ranging in cell doses of 50 to 1000 cells. Thy1⁺CD24⁺CD45− cells collected from passaged tumors were also enriched for tumor-forming cells when injected into recipient mice as shown in panel (b) at doses of 50 to 2000 cells. In contrast, “Not Thy1⁺CD24⁺CD45−” cells from de novo tumors were significantly reduced in tumor forming capacity when injected into recipient mice at cell doses of 1000 to 10,000 cells as shown in panel (c). Similar results were seen when “Not Thy1⁺CD24⁺CD45−” cells from passaged tumors were injected into recipient mice as shown in panel (d).

FIG. 4 shows that normal mammary Thy1.1⁺CD24⁺CD49f⁺CD45⁻ cells contribute to ductal outgrowths when transplanted into Thy1.2 mice. Mammary tissue was dissociated and stained with Thy1.1, CD24, CD49f, CD45 and DAPI for flow cytometry. (a) Mammary tissue cells show heterogeneous CD24 and CD49f expression after selectively gating for CD45− and DAPI− cells. (b) Purity of CD24⁺CD49f⁺CD45− population after cell sorting of CD45−DAPI− cells with gating on Thy1.1⁺ and CD24⁺CD49f⁺ cells. Only cells that were positive for Thy1.1, CD24, and CD49f together were considered Thy1.1⁺CD24⁺CD49f⁺CD45−. As low as 1000 cells produced a new ductal outgrowth that contained Thy1.1 cells when injected into the cleared fat pads of recipient mice. (c) Purity of the “Not Thy1.1⁺CD24⁺CD49f⁺CD45−” population after cell sorting of (a) using gating that selects cells not expressing both CD24 and CD49f. These cells are considered “Not Thy1.1⁺CD24⁺CD49f⁺CD45−”, and failed to produce ductal outgrowths when 10,000 cells were injected. (d) Ductal outgrowths after injection of Thy1.1⁺CD24⁺CD49f⁺CD45− donor cells into recipient mice cleared of the mammary fat pad contained Thy1.1 cells. Donor cells contributed an average of 1.96±1.10% to the reconstituted tissue.

FIG. 5 shows that mouse tumorigenic cells have decreased cytokeratin 19 (CK19) and have similar array profiles as human breast cancer tumorigenic cells. (a) Results of real-time PCR for CK19 on Wnt-1 tumors showed greater than a 100-fold increase of mRNA in the non-tumorigenic “Not CD24⁺Thy+CD45−” cells as compared to the tumorigenic CD24⁺Thy⁺CD45− cells. (b) Hierarchical cluster analysis of tumorigenic and non-tumorigenic cells of both human and mouse breast cancers. A hierarchical cluster analysis shows that the mouse tumorigenic cells cluster with the human tumorigenic cells and the mouse nontumorigenic cells cluster with the human non-tumorigenic cells. Tumorigenic clustering is shown in the first 5 columns with three different mouse tumors, an average of the three mouse tumors, and an average of 6 human tumor samples shown. The next 5 columns show non-tumorigenic clustering, again with three different mouse tumors, an average of the three mouse tumors, and an average of 6 human tumor samples are shown.

FIG. 6 shows that breast cancer stem cell gene signatures can predict survival rates in different types of tumors. In each dataset, tumors were separated into two groups based on the correlation value with the human breast cancer tumorigenic cell gene signature. An average correlation value of all tumors within one dataset was used as the threshold. Using this threshold, roughly equal numbers of tumors were in each group. (a) Kaplan-Meier survival curves of the two groups of patients with tumors in 62 early stage lung cancers (Bhattacharjee, A. et al., Proc Natl Acad Sci U S A 98:13790-5 (2001)). Overall survival was the clinical end point. Patients with a tumor gene expression patterns more similar to the tumorigenic gene signature had a worse outcome than patients with tumor gene expression patterns less similar to the tumorigenic gene signature. (b) Kaplan-Meier survival curves of two groups of patients with 60 medulloblastomas (Ramaswamy, S. et al., Nat Genet 33:49-54 (2003)) using overall survival of these patients as the clinical end point. Patients with tumor gene expression pattern more similar to the tumorigenic gene signature had worse outcome than patients with tumor gene expression pattern less similar to the tumorigenic gene signature. (c) Kaplan-Meier survival curves of two groups of patients with tumors in 21 prostate cancers (Singh, D. et al., Cancer Cell 1:203-9 (2002)) using time to PSA relapse after radical prostatectomy as the clinical end point. Patients with tumor gene expression patterns more similar to the tumorigenic gene signature had a worse outcome than patients with tumor gene expression pattern less similar to the tumorigenic gene signature.

FIG. 7 shows the Thy1.1 staining patterns of MMTV-Wnt-1 breast tumors as compared to normal mouse breast tissue, and thymus. Hematoxylin and eosin staining of (a) a normal mouse and (c) a Wnt-1 tumor. Thy1 staining in (b) normal breast, (d) a Wnt-1 tumor, and (e) a thymus (positive control). Arrows highlight the few cells within the Wnt-1 tumor which stain for Thy-1.

FIG. 8 shows that in the MMTV Wnt-1 tumor, the majority of CD24⁺Thy⁺ cells are also CD49f⁺. (a) Flow cytometry analysis of dissociated MMTV-Wnt-1 tumors stained with CD24 and Thy1. (b) After gating on Thy1⁺CD24⁺CD45−, the majority of Thy1⁺CD24⁺CD45-cells were shown to be also CD49f⁺.

FIG. 9 shows that breast cancer stem cell gene signatures can predict survival rates in breast cancer. The genes of the gene signature are differentially expressed between mouse TG and NTG cells and also present on the NKI database numbered 59. Using the list of differentially expressed mouse genes, we were able to predict survival and metastasis free survival of the 295 breast cancer patients in the Netherlands Cancer Institute (NKI). A Pearson correlation was calculated between gene signature and each tumor, and tumors were separated into two groups (good prognosis and poor prognosis) based on their correlation value. The good prognosis group contained 102 patients and the poor prognosis contained 193 patients. Kaplan-Meier survival curves of two groups of patients were calculated using overall survival (a) or metastasis (b) as the clinical end point. Survival and distant metastasis free survival at 12 years was 86% and 73% respectively for the good prognosis group and 59% and 54% respectively for the poor prognosis. Respective p-values are shown.

FIG. 10: Estrogen receptor positive patients can be divided into good and poor prognosis. The 59 genes mentioned in FIG. 9 were used to analyze the NKI database patients who were estrogen receptor positive (ER+). A Pearson correlation was calculated between gene signature and each tumor, and tumors were separated into two groups (good prognosis and poor prognosis) based on their correlation value. Of the 206 patients, 101 were in the good prognostic group and 125 were in the poor prognostic group. Patients in the good prognosis group had a 12 year survival of 86% (a) and 72% were metastasis free (b) as compared to those in the poor prognosis group who had a 12 year survival of 68% (a) and 57% metastasis free survival (b). Respective p-values are shown.

### DETAILED DESCRIPTION OF THE INVENTION

This invention is based on the discovery of solid tumor stem cells (also referred to as cancer stem cells from a solid tumor) as a distinct and limited subset of cells within the heterogeneous cell population of established solid tumors. These cancer stem cells share the properties of normal stem cells in that they extensively proliferate and efficiently give rise both to additional solid tumor stem cells (self-renewal) and to the majority of tumor cells of a solid tumor that lack tumorigenic potential. Identification of cancer stem cells from solid tumors relied on their expression of a unique pattern of cell-surface receptors that could be used to isolate them from the bulk of non-tumorigenic tumor cells and on the assessment of their properties of self-renewal and proliferation in culture and in xenograft animal models. An ESA⁺; CD44⁺; CD24−/low; Lineage- population greater than 50-fold enriched for the ability to form tumors relative to unfractionated tumor cells was discovered (Al-Hajj et al., 2003; U.S. Appl. Pub. Nos. 2002/0119565 and 2004/0037815; International Appl. PCT/US02/38181; each of which are herein incorporated by reference).

The present invention provides an isolated population of murine solid tumor stem cells. The population comprises at least 75% solid tumor stem cells and less than 25% solid tumor cells. The present invention relates to compositions and methods for treating, characterizing and diagnosing cancer. In particular, the present invention provides gene expression profiles associated with solid tumor stem cells, as well as novel markers useful for the diagnosis, characterization, and treatment of murine solid tumor stem cells. These markers can also be used as prognostic indicators for patient survival. Suitable markers that can be targeted (e.g. for diagnostic or therapeutic purposes) are the genes and peptides encoded by the genes that are differentially expressed in solid tumor stem cells as shown in Table 1. The differentially expressed genes, and the peptides encoded thereby, can be detected (e.g. quantitatively) in order to identify the presence of solid tumor stem cells, and to determine and screen molecules suitable for reducing the proliferation (or killing), interfering with self-renewal pathways, or interfering with survival pathways of any solid tumor stem cells that are present. The differentially expressed genes, and peptides encoded thereby, shown in Table 1 are also useful for generating therapeutic agents targeted to one or more of these markers (e.g. to inhibit or promote the activity of the marker).

The present invention also provides solid tumor stem cells that differentially express from other cells one or more of the markers provided in Table 1. The solid tumor stem cells can be human, mouse or other animal. The expression can be either to a greater extent or to a lesser extent. The other cells can be selected from normal cells, hematopoietic stem cells, acute myelogenous leukemia (AML) stem cells, or any other class of cells.

The invention provides a method of selecting cells of a population, which results in a purified population of solid tumor stem cells (e.g. from a patient to select or test therapeutic agents are some for the patient). The present invention also provides a method of selecting a purified population of tumor cells other than solid tumor stem cells, such as a population of non-tumorigenic (NTG) tumor cells. The present invention provides methods of raising antibodies to the selected cells. The invention also provides diagnostic methods using the selected cells. Furthermore, the invention also provides therapeutic methods, where the therapeutic is directed to a solid tumor stem cell (e.g. directed to one of the stem cells cancer markers identified herein directly or indirectly).

The invention provides in vivo and in vitro assays of solid tumor stem cell function and cell function by the various populations of cells isolated from a solid tumor.

The invention provides methods for using the various populations of cells isolated from a solid tumor (such as a population of cells enriched for solid tumor stem cells) to identify factors influencing solid tumor stem cell proliferation. By the methods of the present invention, one can characterize the phenotypically heterogeneous populations of cells within a solid tumor. In particular, one can identify, isolate, and characterize a phenotypically distinct cell population within a tumor having the stem cell properties of extensive proliferation and the ability to give rise to all other tumor cell types. Solid tumor stem cells are the tumorigenic cells that are capable of re-establishing a tumor following treatment.

In some embodiments, the present invention provides methods for screening for anti-cancer agents; for the testing of anti-cancer therapies; for the development of drugs targeting novel pathways; for the identification of new anti-cancer therapeutic targets; the identification and diagnosis of malignant cells in pathology specimens; for the testing and assaying of solid tumor stem cell drug sensitivity; for the measurement of specific factors that predict drug sensitivity; and for the screening of patients (e.g., as an adjunct for mammography).

The present invention further provides a cancer stem cell gene signature comprising cancer stem cell markers that are predictive of clinical outcome including metastasis and overall survival. This cancer stem cell signature is shown in Table 1 and is shown to be predictive of a poor prognosis. In some embodiments of the present invention, the cancer stem cell signatures are used clinically to classify tumors as low or high risk and to assign a tumor to a low or high risk category. The cancer stem cell signatures can further be used to provide a diagnosis, prognosis, and select a therapy based on the classification of a tumor as low or high risk as well as to monitor the diagnosis, prognosis, and/or therapy over time. In another embodiment, the cancer stem cell signatures can be used experimentally to test and assess lead compounds including, for example, small molecules, siRNAs, and antibodies for the treatment of cancer.

In certain embodiments a tumor cell profile is detected by quantifying expression levels of polynucleotides by, for example, RT-PCR. The polynucleotides selected for quantification by RT-PCR are those polynucleotides comprising the cancer stem cell signature. Alternatively the tumor cell profiles are detected by quantifying expression levels of proteins by, for example, quantitative immunofluorescence or ELISA. The proteins selected for quantification are those proteins encoded by genes comprising the cancer stem cell signature. In a some embodiment, a tumor cell profile is detected by microarray analysis using microarrays that comprise a cancer stem cell gene signature. These microarrays can detect the presence of a tumor cell profile by expression levels of polynucleotides, for example mRNA, in a patient sample or, alternatively, by expression levels of proteins in a patient sample using, for example, antibodies. In another some embodiment, a tumor cell profile is detected by real-time PCR using primer sets that specifically amplify the genes comprising the cancer stem cell signature. In other embodiments of the invention, microarrays are provided that contain polynucleotides or proteins (i.e. antibodies) that detect the expression of a cancer stem cell signature for use in prognosis.

To better elucidate the biology of tumorigenic cancer cells, mouse models were used since the study of cancer stem cells in humans is limited by the difficulty in investigating the molecular regulation of human organ development in vivo. In mice, transplantation assays to identify cells with regenerative potential have been established and have demonstrated that in the murine mammary gland, stem and progenitor cells within the terminal end buds and mammary ducts give rise to the epithelial components of the mammary gland, specifically luminal epithelial cells and contractile myoepithelial cells (Smith, G. H. and Boulanger, C. A. Cell Prolif 36 Suppl 1:3-15 (2003); (Smith, G. H. and Chepko, G. Microsc Res Tech 52:190-203 (2001)). The luminal epithelial cells line the ducts and alveoli, and myoepithelial cells are typically located between the luminal cells and basement membrane.

MMTV-Wnt-1 transgenic mice, which develop mammary tumors as a consequence of activation of β-catenin signaling, were used to help identify a population of cells with cancer tumorigenic cell properties. In the MMTV-Wnt-1 transgenic mice, a subpopulation of breast tumor cells from these mice has been identified based on cell surface marker expression. These markers include Thy1, CD24, CD49f and CD45. Cell populations that are highly enriched for cancer cells able to establish tumors when transplanted into syngeneic recipients. More specifically, tumor cells expressing Thy1⁺CD24⁺CD49f⁺CD45⁻ are capable of regenerating tumors of the same cellular heterogeneity as the original tumor even when injected as low as 50 cells into the mammary fat pad of recipient mice. In addition, these cells retain their ability to replicate and differentiate after serial transplantation.

Interestingly, Thy1⁺CD24⁺CD49f⁺CD45⁻ cells isolated from normal murine mammary tissue were capable of regenerating mammary tissue in vivo when implanted into the recipient mice cleared of the mammary fat pad. This strongly suggests that the phenotype of the tumorigenic cancer cells is similar to that of normal mammary epithelial cells with duct-regenerative capacity. Using microarray analysis, a strong correlation between mouse breast cancer tumorigenic cells and human breast cancer tumorigenic cells is shown implying that there are common molecular pathways that establish tumorigenicity. The present invention shows for the first time that the tumorigenic gene signature has clinical significance in being able to predict patient survival and disease relapse in other human cancers.

### Definitions

To facilitate an understanding of the present invention, a number of terms and phrases are defined below:

The term “antibody” as used herein refers to immunoglobulin molecules and immunologically active portions of immunoglobulin (Ig) molecules. Such antibodies include, but are not limited to, polyclonal, monoclonal, chimeric, single chain, Fab, Fab′ and Fab′₂ fragments, Fv, and an Fab expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of antibody species.

It has been shown that fragments of an antibody can perform the function of binding antigens. As used herein “antigen binding fragments” include, but are not limited to: (i) the Fab fragment consisting of V_{L}, V_{H}, C_{L} and C_{H}1 domains; (ii) the Fd fragment consisting of the V_{H} and C_{H}1 domains; (iii) the Fv fragment consisting of the V_{L} and V_{H} domains of a single antibody; (iv) the dAb fragment (Ward, E. S. et al., Nature 341:544-546 (1989)) which consists of a V_{H} domain; (v) isolated CDR regions; (vi) F(ab′)₂ fragments (vii) single chain Fv molecules (scFv), wherein a V_{H} domain and a V_{L} domain are linked by a peptide linker which allows the two domains to associate to form an antigen binding site (Bird, et al., Science 242:423-426 (1988); Huston et al., Proc. Natl. Acad. Sci. USA 85:5879-5883 (1988)); (viii) bispecific single chain Fv dimers (PCT/US92/09965) and (ix) diabodies, multivalent or multispecific fragments constructed by gene fusion (WO94/13804; P. Holliger et al., Proc. Natl. Acad. Sci. USA 90:6444-6448 (1993).

“Enriched”, as in an enriched population of cells, can be defined phenotypically based upon the increased number of cells having a particular marker (e.g. as shown in Table 1) in a fractionated set of cells as compared with the number of cells having the marker in the unfractionated set of cells. However, the term “enriched” can be defined functionally by tumorigenic function as the minimum number of cells that form tumors at limit dilution frequency in test mice. For example, if 500 tumor stem cells form tumors in 63% of test animals, but 5000 unfractionated tumor cells are required to form tumors in 63% of test animals, then the solid tumor stem cell population is 10-fold enriched for tumorigenic activity. The stem cell cancer markers of the present invention can be used to generate enriched populations of cancer stem cells. In some embodiments, the stem cell population is enriched at least 1.4 fold relative to unfractioned tumor cells (e.g. 1.4 fold, 1.5 fold, 2 fold, 5 fold, . . . , 20 fold, . . . ).

“Isolated” in regard to cells, refers to a cell that is removed from its natural environment (such as in a solid tumor) and that is isolated or separated, and is at least about 30%, 50%, 75% free, or about 90% free, from other cells with which it is naturally present, but which lack the marker based on which the cells were isolated. The stem cell cancer markers of the present invention can be used to generate isolated populations of cancer stem cells.

As used herein, the terms “low levels”, “decreased levels”, “low expression”, “reduced expression” or “decreased expression” in regards to gene expression are used herein interchangeably to refer to expression of a gene in a cell or population of cells, particularly a cancer stem cell or population of cancer stem cells, at levels less than the expression of that gene in a second cell or population of cells, for example normal breast epithelial cells. “Low levels” of gene expression refers to expression of a gene in a cancer stem cell or population of cancer stem cells at levels: 1) half that or below expression levels of the same gene in normal breast epithelial cells and 2) at the lower limit of detection using conventional techniques. “Low levels” of gene expression can be determined by detecting decreased to nearly undetectable amounts of a polynucleotide (mRNA, cDNA, etc.) in cancer stem cells compared to normal breast epithelium by, for example, quantitative RT-PCR or microarray analysis. Alternatively “low levels” of gene expression can be determined by detecting decreased to nearly undetectable amounts of a protein in cancer stem cells compared to normal breast epithelium by, for example, ELISA, Western blot, quantitative immunofluorescence, etc.

The terms “high levels”, “increased levels”, “high expression”, “increased expression” or “elevated levels” in regards to gene expression are used herein interchangeably to refer to expression of a gene in a cell or population of cells, particularly a cancer stem cell or population of cancer stem cells, at levels higher than the expression of that gene in a second cell or population of cells, for example normal breast epithelial cells. “Elevated levels” of gene expression refers to expression of a gene in a cancer stem cell or population of cancer stem cells at levels twice that or more of expression levels of the same gene in normal breast epithelial cells. “Elevated levels” of gene expression can be determined by detecting increased amounts of a polynucleotide (mRNA, cDNA, etc.) in cancer stem cells compared to normal breast epithelium by, for example, quantitative RT-PCR or microarray analysis. Alternatively “elevated levels” of gene expression can be determined by detecting increased amounts of a protein in cancer stem cells compared to normal breast epithelium by, for example, ELISA, Western blot, quantitative immunofluorescence, etc.

The term “undetectable levels” or “loss of expression” in regards to gene expression as used herein, refers to expression of a gene in a cell or population of cells, particularly a cancer stem cell or population of cancer stem cells, at levels that cannot be distinguished from background using conventional techniques such that no expression is identified. “Undetectable levels” of gene expression can be determined by the inability to detect levels of a polynucleotide (mRNA, cDNA, etc.) in cancer stem cells above background by, for example, quantitative RT-PCR or microarray analysis. Alternatively “undetectable levels” of gene expression can be determined by the inability to detect levels of a protein in cancer stem cells above background by, for example, ELISA, Western blot, immunofluorescence, etc.

As used herein, the term “receptor binding domain” refers to any native ligand for a receptor, including cell adhesion molecules, or any region or derivative of such native ligand retaining at least a qualitative receptor binding ability of a corresponding native ligand.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The term “epitope” as used herein refers to that portion of an antigen that makes contact with a particular antibody.

When a protein or fragment of a protein is used to immunize a host animal, numerous regions of the protein can induce the production of antibodies which bind specifically to a given region or three-dimensional structure on the protein; these regions or structures are referred to as “antigenic determinants”. An antigenic determinant can compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer can also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental exposure, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers can be characterized by the identification of the expression of one or more cancer marker genes, including but not limited to, the cancer markers disclosed herein.

The terms “cancer stem cell”, “tumor stem cell”, or “solid tumor stem cell” are used interchangeably herein and refer to a population of cells from a solid tumor that: (1) have extensive proliferative capacity; (2) are capable of asymmetric cell division to generate one or more kinds of differentiated progeny with reduced proliferative or developmental potential; and (3) are capable of symmetric cell divisions for self-renewal or self-maintenance. These properties of “cancer stem cells”, “tumor stem cells” or “solid tumor stem cells” confer on those cancer stem cells the ability to form palpable tumors upon serial transplantation into an immunocompromised mouse compared to the majority of tumor cells that fail to form tumors. Cancer stem cells undergo self-renewal versus differentiation in a chaotic manner to form tumors with abnormal cell types that can change over time as mutations occur. The solid tumor stem cells of the present invention differ from the “cancer stem line” provided by U.S. Pat. No. 6,004,528. In that patent, the “cancer stem line” is defined as a slow growing progenitor cell type that itself has few mutations but which undergoes symmetric rather than asymmetric cell divisions as a result of tumorigenic changes that occur in the cell's environment. This “cancer stem line” hypothesis thus proposes that highly mutated, rapidly proliferating tumor cells arise largely as a result of an abnormal environment, which causes relatively normal stem cells to accumulate and then undergo mutations that cause them to become tumor cells. U.S. Pat. No. 6,004,528 proposes that such a model can be used to enhance the diagnosis of cancer. The solid tumor stem cell model is fundamentally different than the “cancer stem line” model and as a result exhibits utilities not offered by the “cancer stem line” model. First, solid tumor stem cells are not “mutationally spared”. The “mutationally spared cancer stem line” described by U.S. Pat. No. 6,004,528 can be considered a pre-cancerous lesion, while the solid tumor stem cells described by this invention are cancer cells that themselves contain the mutations that are responsible for tumorigenesis. That is, the solid tumor stem cells (“cancer stem cells”) of the invention would be included among the highly mutated cells that are distinguished from the “cancer stem line” in U.S. Pat. No. 6,004,528. Second, the genetic mutations that lead to cancer can be largely intrinsic within the solid tumor stem cells as well as being environmental. The solid tumor stem cell model predicts that isolated solid tumor stem cells can give rise to additional tumors upon transplantation (thus explaining metastasis) while the “cancer stem line” model would predict that transplanted “cancer stem line” cells would not be able to give rise to a new tumor, since it was their abnormal environment that was tumorigenic. Indeed, the ability to transplant dissociated, and phenotypically isolated human solid tumor stem cells to mice (into an environment that is very different from the normal tumor environment), where they still form new tumors, distinguishes the present invention from the “cancer stem line” model. Third, solid tumor stem cells likely divide both symmetrically and asymmetrically, such that symmetric cell division is not an obligate property. Fourth, solid tumor stem cells can divide rapidly or slowly, depending on many variables, such that a slow proliferation rate is not a defining characteristic.

As used herein “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor. These properties of self-renewal and proliferation to generate all other tumor cells confer on the cancer stem cells of this invention the ability to form palpable tumors upon serial transplantation into an immunocompromised mouse compared to the majority of tumor cells that are unable to form tumors upon the serial transplantation. Tumor cells, i.e. non-tumorigenic tumor cells, may form a tumor upon transplantation into an immunocompromised mouse a limited number of times (for example one or two times) after obtaining the tumor cells from a solid tumor.

As used herein, the terms “stem cell cancer marker(s)”, “cancer stem cell marker(s)”, “tumor stem cell marker(s)”, or “solid tumor stem cell marker(s)” refer to a gene or genes or a protein, polypeptide, or peptide expressed by the gene or genes whose expression level, alone or in combination with other genes, is correlated with the presence of tumorigenic cancer cells compared to non-tumorigenic cells. The correlation can relate to either an increased or decreased expression of the gene (e.g. increased or decreased levels of mRNA or the peptide encoded by the gene).

As used herein, the terms “unfractionated tumor cells”, “presorted tumor cells”, “bulk tumor cells”, and their grammatical equivalents are used interchangeably to refer to a tumor cell population isolated from a patient sample (e.g. a tumor biopsy or pleural effusion) that has not been segregated, or fractionated, based on cell surface marker expression.

As used herein, the terms “non-ESA+CD44+ tumor cells”, “non-ESA+44+”, “sorted non-tumorigenic tumor cells”, “non-tumorigenic tumor cells,” “non-stem cells,” “tumor cells” and their grammatical equivalents are used interchangeably to refer to a tumor population from which the cancer stem cells of this invention have been segregated, or removed, based on cell surface marker expression.

“Gene expression profile” refers to identified expression levels of at least one polynucleotide or protein expressed in a biological sample.

A “gene profile,” “gene pattern,” “expression pattern” or “expression profile” refers to a specific pattern of gene expression that provides a unique identifier of a biological sample, for example, a breast or colon cancer pattern of gene expression obtained by analyzing a breast or colon cancer sample will be referred to as a “breast cancer gene profile” or a “colon cancer expression pattern”. “Gene patterns” can be used to diagnose a disease, make a prognosis, select a therapy, and/or monitor a disease or therapy after comparing the gene pattern to a cancer stem cell gene signature.

The terms “cancer stem cell gene signature”, “tumor stem cell gene signature”, “cancer stem cell signature”, “tumor stem cell signature”, “tumorigenic gene signature”, and “TG gene signature” are used interchangeably herein to refer to gene signatures comprising genes differentially expressed in cancer stem cells compared to other cells or population of cells, for example normal breast epithelial tissue. In some embodiments the cancer stem cell gene signatures comprise genes differentially expressed in cancer stem cells versus normal breast epithelium by a fold change, for example by 2 fold reduced and/or elevated expression, and further limited by using a statistical analysis such as, for example, by the P value of a t-test across multiple samples. In another some embodiment, the genes differentially expressed in cancer stem cells are divided into cancer stem cell gene signatures based on the correlation of their expression with a chosen gene in combination with their fold or percentage expression change. Most some cancer stem cell signatures are predictive both retrospectively and prospectively of an aspect of clinical variability, including but not limited to metastasis and death.

As used herein, the term “a reagent that specifically detects expression levels” refers to reagents used to detect the expression of one or more genes (e.g., including but not limited to, the cancer markers of the present invention). Examples of suitable reagents include but are not limited to, nucleic acid probes capable of specifically hybridizing to the gene of interest, aptamers, PCR primers capable of specifically amplifying the gene of interest, and antibodies capable of specifically binding to proteins expressed by the gene of interest. Other non-limiting examples can be found in the description and examples below.

As used herein, the term “detecting a decreased or increased expression relative to non-cancerous control” refers to measuring the level of expression of a gene (e.g., the level of mRNA or protein) relative to the level in a non-cancerous control sample. Gene expression can be measured using any suitable method, including but not limited to, those described herein.

As used herein, the term “detecting a change in gene expression in a cell sample in the presence of the test compound relative to the absence of the test compound” refers to measuring an altered level of expression (e.g., increased or decreased) in the presence of a test compound relative to the absence of the test compound. Gene expression can be measured using any suitable method.

As used herein, the term “instructions for using the kit for detecting cancer in the subject” includes instructions for using the reagents contained in the kit for the detection and characterization of cancer in a sample from a subject.

As used herein, “providing a diagnosis” or “diagnostic information” refers to any information that is useful in determining whether a patient has a disease or condition and/or in classifying the disease or condition into a phenotypic category or any category having significance with regards to the prognosis of or likely response to treatment (either treatment in general or any particular treatment) of the disease or condition. Similarly, diagnosis refers to providing any type of diagnostic information, including, but not limited to, whether a subject is likely to have a condition (such as a tumor), information related to the nature or classification of a tumor, information related to prognosis and/or information useful in selecting an appropriate treatment. Selection of treatment can include the choice of a particular chemotherapeutic agent or other treatment modality such as surgery, radiation, etc., a choice about whether to withhold or deliver therapy, etc.

As used herein, the terms “providing a prognosis”, “prognostic information”, or “predictive information” refer to providing information regarding the impact of the presence of cancer (e.g., as determined by the diagnostic methods of the present invention) on a subject's future health (e.g., expected morbidity or mortality, the likelihood of getting cancer, and the risk of metastasis).

The term “low risk” in regards to tumors or to patients diagnosed with cancer refers to a tumor or patient with a lower probability of metastasis and/or lower probability of causing death or dying within about five years of first diagnosis than all the tumors or patients within a given population.

The term “high risk” in regards to tumors or to patients diagnosed with cancer refers to a tumor or patient with a higher probability of metastasis and/or higher probability of causing death or dying within about five years of first diagnosis than all the tumors or patients within a given population.

As used herein, the terms “biopsy tissue”, “patient sample”, “tumor sample”, and “cancer sample” refer to a sample of cells, tissue or fluid that is removed from a subject for the purpose of determining if the sample contains cancerous tissue, including cancer stem cells or for determining gene expression profile of that cancerous tissue. In some embodiment, biopsy tissue or fluid is obtained because a subject is suspected of having cancer. The biopsy tissue or fluid is then examined for the presence or absence of cancer, cancer stem cells, and/or cancer stem cell gene signature expression.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5- carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl- 2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns can contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g, mRNA, rRNA, tRNA, or snRNA) through “transcription” of the gene (e.g., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (e.g., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene can also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region can contain regulatory sequences such as promoters and enhancers that control or influence the transcription of the gene. The 3′ flanking region can contain sequences that direct the termination of transcription, post-transcriptional cleavage and polyadenylation.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs can also contain additional sequences; non- limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene can be endogenous or exogenous to the organism, present integrated into a chromosome or present in a transfection vector that is not integrated into the genome. The expression of the gene is either completely or partially inhibited. RNAi can also be considered to inhibit the function of a target RNA; the function of the target RNA can be complete or partial.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region can be present in a cDNA, genomic DNA or RNA form. When present in a DNA form, the oligonucleotide or polynucleotide can be single-stranded (i.e., the sense strand) or double- stranded. Suitable control elements such as enhancers/promoters, splice junctions, polyadenylation signals, etc. can be placed in close proximity to the coding region of the gene if needed to permit proper initiation of transcription and/or correct processing of the primary RNA transcript. Alternatively, the coding region utilized in the expression vectors of the present invention can contain endogenous enhancers/promoters, splice junctions, intervening sequences, polyadenylation signals, etc. or a combination of both endogenous and exogenous control elements.

As used herein, the term “portion” when in reference to a nucleotide sequence (as in “a portion of a given nucleotide sequence”) refers to fragments of that sequence. The fragments can range in size from four nucleotides to the entire nucleotide sequence minus one nucleotide (10 nucleotides, 20, 30, 40, 50, 100, 200, etc.).

The phrases “hybridizes”, “selectively hybridizes”, or “specifically hybridizes” refer to the binding or duplexing of a molecule only to a particular nucleotide sequence under stringent hybridization conditions when that sequence is present in a complex mixture (e.g., a library of DNAs or RNAs). See, e.g., Andersen (1998) Nucleic Acid Hybridization Springer-Verlag; Ross (ed. 1997) Nucleic Acid Hybridization Wiley.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency hybridization, a positive signal is at least two times background, or 10 times background hybridization. Exemplary high stringency or stringent hybridization conditions include: 50% formamide, 5×SSC, and 1% SDS incubated at 42° C. or 5×SSC and 1% SDS incubated at 65° C., with a wash in 0.2×SSC and 0.1% SDS at 65° C. For PCR, a temperature of about 36° C. is typical for low stringency amplification, although annealing temperatures can vary from about 32° C. and 48° C. depending on primer length. For high stringency PCR amplification, a temperature of about 62° C. is typical, although high stringency annealing temperatures can range from about 50-65° C., depending on the primer length and specificity. Typical cycle conditions for both high and low stringency amplifications include a denaturation phase of 90-95° C. for 30-120 seconds, an annealing phase lasting 30-120 seconds, and an extension phase of about 72° C. for 1-2 minutes.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non- isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide can be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide can be single-stranded), but can contain both the sense and anti-sense strands (i.e., the oligonucleotide or polynucleotide can be double-stranded).

“Amino acid sequence” and terms such as “polypeptide”, “protein”, or “peptide” are not meant to limit the amino acid sequence to the complete, native amino acid sequence associated with the recited protein molecule.

As used herein the term “portion” when in reference to a protein (as in “a portion of a given protein”) refers to fragments of that protein. The fragments can range in size from four amino acid residues to the entire amino acid sequence minus one amino acid.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and can include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 1.5-fold higher (or greater) than that observed in a given tissue in a control or non- transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reactions that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples can be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “subject diagnosed with a cancer” refers to a subject who has been tested and found to have cancerous cells. The cancer can be diagnosed using any suitable method, including but not limited to, biopsy, x-ray, blood test, and the diagnostic methods of the present invention.

In some embodiments, the discrimination between cells based upon the detected expression of cell surface markers is by comparing the detected expression of the cell surface marker as compared with the mean expression by a control population of cells. For example, the expression of a marker on a solid tumor stem cell can be compared to the mean expression of the marker by the other cells derived from the same tumor as the solid tumor stem cell. Other methods of discriminating among cells by marker expression include methods of gating cells by flow cytometry based upon marker expression (see, Givan A, Flow Cytometry: First Principles, (Wiley-Liss, New York, 1992); Owens M A & Loken M R., Flow Cytometry: Principles for Clinical Laboratory Practice, (Wiley-Liss, New York, 1995)).

Solid tumor stem cell positive markers may also be present on cells other than solid tumor stem cells. Solid tumor stem cell negative markers may also be absent from cells other than solid tumor stem cells. While it is rare to identify a single marker that identifies a stem cell, it has often been possible to identify combinations of positive and negative markers that uniquely identify stem cells and allow their substantial enrichment in other contexts. Morrison et al., Cell 96(5): 737-49 (1999); Morrison et al., Proc. Natl. Acad. Sci. USA 92(22): 10302-6 (1995); Morrison & Weissman, Immunity 1(8): 661-73 (1994).

A “combination of reagents” is at least two reagents that bind to cell surface markers either present (positive marker) or not present (negative marker) on the surfaces of solid tumor stem cells, or to a combination of positive and negative markers. The use of a combination of antibodies specific for solid tumor stem cell surface markers results in the method of the invention being useful for the isolation or enrichment of solid tumor stem cells from a variety of solid tumors, including sarcomas, ovarian cancers, and breast tumors. Guidance to the use of a combination of reagents can be found in PCT patent application WO 01/052143 (Morrison & Anderson), incorporated by reference.

By selecting for phenotypic characteristics among the cells obtained from a solid tumor, solid tumor stem cells can be isolated from any animal solid tumor, particularly any mammalian solid tumor. It will be appreciated that, taking into consideration factors such as a binding affinities, that antibodies that recognize species-specific varieties of markers are used to enrich for and select solid tumor stem cells. Antibodies that recognize the species-specific varieties of Thy1, CD24, CD49f, CD45 and other markers will be used to enrich for or isolate solid tumor stem cells from that species (for example, antibody to a mouse CD45 for mouse solid tumor stem cells, antibody to a monkey CD24 for monkey solid tumor stem cells, etc.).

Therapeutic Aspects of the Invention

A corollary to the solid tumor stem cell model of the invention is that, to effectively treat cancer and achieve higher cure rates, anti-cancer therapies must be directed against solid tumor stem cells. Since current therapies are directed against the bulk population, they may be ineffective at eradicating solid tumor stem cells. The limitations of current cancer therapies derive from their inability to effectively kill solid tumor stem cells. The identification of solid tumor stem cells permits the specific targeting of therapeutic agents to this cell population, resulting in more effective cancer treatments. This concept would fundamentally change our approach to cancer treatment.

One of the major problems in identifying new cancer therapeutic agents is determining which of the myriad of genes identified in large scale sequencing projects are the most clinically important drug targets. This is made especially difficult since solid tumors consist of a mixture of a many types of normal cells and a heterogeneous population of tumor cells. One way to reduce the complexity is to make cDNA after microdissection of solid tumors to enrich for tumor cells. This technique is based on the assumption that the pathologist dissecting out the tumor cells can predict which cells are tumorigenic based upon appearance. However, cells can be morphologically similar and yet remain functionally heterogeneous. Moreover, cells obtained by microdissection are not viable and therefore the functional properties of such cells cannot be tested or verified.

In vitro proliferation of solid tumor stem cells. Cells can be obtained from solid tumor tissue by dissociation of individual cells. Tissue from a particular tumor is removed using a sterile procedure, and the cells are dissociated using any method known in the art (see, Sambrook et al., Molecular Cloning. A Laboratory Manual (Cold Spring Harbor Press, Cold Spring Harbor, N.Y., 1989); Current Protocols in Molecular Biology, Ausubel et al., eds., (Wiley Interscience, New York, 1993), and Molecular Biology LabFax, Brown, ed. (Academic Press, 1991)), including treatment with enzymes such as trypsin, collagenase and the like, or by using physical methods of dissociation such as with a blunt instrument. Methods of dissociation are optimized by testing different concentrations of enzymes and for different periods of time, to maximize cell viability, retention of cell surface markers, and the ability to survive in culture (Worthington Enzyme Manual, Von Worthington, ed. (Worthington Biochemical Corporation, 2000). Dissociated cells are centrifuged at low speed, from about 200 rpm to about 2000 rpm, usually about 1000 rpm (210 g), and then resuspended in culture medium. For guidance to methods for cell culture, see Spector et al., Cells: A Laboratory Manual (Cold Spring Harbor Press, Cold Spring Harbor, N.Y., 1998).

The dissociated tumor cells can be placed into any known culture medium capable of supporting cell growth, including HEM, DMEM, RPMI, F-12, and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. However, some embodiments for proliferation of solid tumor stem cells are to use a defined, low-serum culture medium. In some embodiments, a culture medium for solid tumor stem cells is a defined culture medium comprising a mixture of Ham's F12, 2% fetal calf serum, and a defined hormone and salt mixture, either insulin, transferrin, and selenium or B27 supplement. Brewer et al., J. Neuroscience Res. 35: 567 (1993).

The culture medium can be a chemically defined medium that is supplemented with fetal bovine serum or chick embryo extract (CEE) as a source of mitogens and survival factors to allow the growth of tumor stem cells in culture. Other serum-free culture medium containing one or more predetermined growth factors effective for inducing stem cell proliferation, such as N2 supplement or B27 supplement, known to those of skill in the art can be used to isolate and propagate solid tumor stem cells from other bird and mammalian species, such as human. See, U.S. Pat. Nos. 5,750,376, 5,851,832, and 5,753,506; Atlas et al., Handbook of Microbiological Media (CRC Press, Boca, Raton, La., 1993); Freshney, Cutler on Animal Cells, A Manual of Basic Technique, 3d Edition (Wiley-Liss, New York, 1994), all incorporated herein by reference.

The culture medium for the proliferation of solid tumor stem cells thus supports the growth of solid tumor stem cells and the proliferated progeny. The “proliferated progeny” are undifferentiated tumor cells, including solid tumor stem cells, since solid tumor stem cells have a capability for extensive proliferation in culture.

Conditions for culturing should be close to physiological conditions. The pH of the culture medium should be close to physiological pH, such as from pH 6-8, more about pH 7 to 7.8, or pH 7.4. Physiological temperatures range from about 30° C. to 40° C. Cells can be cultured at temperatures from about 32° C. to about 38° C., or from about 35° C. to about 37° C. Similarly, cells may be cultured in levels of 0₂ that are comparatively reduced relative to 0₂ concentrations in air, such that the 0₂ concentration is comparable to physiological levels (1-6%), rather than 20% 0₂ in air.

Solid tumor stem cells, once they have been proliferated in vitro, can be analyzed and screened. Solid tumor stem cell proliferated in vitro can also be genetically modified using techniques known in the art (see, below; see also, Sambrook et al., Molecular Cloning: A Laboratory Manual (Cold Spring Harbor Press, Cold Spring Harbor, N.Y., 1989); Current Protocols in Molecular Biology, Ausubel et al., eds., (Wiley Interscience, New York, 1993)). The in vitro genetic modification may be more desirable in certain circumstances than in vivo genetic modification techniques when more control over the infection with the genetic material is required.

Solid tumor stem cells and stem cell progeny can be cryopreserved until they are needed by any method known in the art. The cells can be suspended in an isotonic solution, such as a cell culture medium, containing a particular cryopreservant. Such cryopreservants include dimethyl sulfoxide (DMSO), glycerol and the like. These cryopreservants are used at a concentration of 5-15% or 8-10%. Cells are frozen gradually to a temperature of −10° C. to −150° C., −20° C. to −100° C., or −150° C.

Genetic Modification of Solid Tumor Stem Cells and Solid Tumor Stem Cell Progeny

In the undifferentiated state, the solid tumor stem cells rapidly divide and are therefore excellent targets for genetic modification. The term “genetic modification” as used herein refers to the stable or transient alteration of the genotype of a precursor cell by intentional introduction of exogenous DNA. DNA may be synthetic, or naturally derived, and may contain genes, portions of genes, or other useful DNA sequences. The term “genetic modification” as used herein is not meant to include naturally occurring alterations such as that which occurs through natural viral activity, natural genetic recombination, or the like. General methods for the genetic modification of eukaryotic cells are known in the art. See, Sambrook et al., Molecular Cloning: A Laboratory Manual (Cold Spring Harbor Press, Cold Spring Harbor, N.Y., 1989); Current Protocols in Molecular Biology, Ausubel et al., eds., (Wiley Interscience, New York, 1993)).

Many methods for introducing vectors into cells or tissues are available and equally suitable for use with solid tumor stem cells in vivo, in vitro, and ex vivo. Vectors may be introduced into hematopoietic stem cells taken from the patient and clonally propagated. By the method of the invention, such methods are extended to solid tumor stem cells.

“Transformation,” or “genetically modified” as defined herein, describes a process by which exogenous DNA enters and changes a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, viral infection, electroporation, heat shock, lipofection, and particle bombardment. The term “transformed” cells includes stably transformed cells in which the inserted DNA is capable of replication either as an autonomously replicating plasmid or as part of the host chromosome, as well as transiently transformed cells which express the inserted DNA or RNA for limited periods of time.

Genetic manipulation of primary tumor cells has been described previously by Patel et al., Human Gene Therapy 5: 577-584 (1994). Genetic modification of a cell maybe accomplished using one or more techniques well known in the gene therapy field. Mulligan R C, Human Gene Therapy 5: 543-563 (1993). Viral transduction methods may comprise the use of a recombinant DNA or an RNA virus comprising a nucleic acid sequence that drives or inhibits expression of a protein to infect a target cell. A suitable DNA virus for use in the present invention includes but is not limited to an adenovirus (Ad), adeno-associated virus (AAV), herpes virus, vaccinia virus or a polio virus. A suitable RNA virus for use in the present invention includes but is not limited to a retrovirus or Sindbis virus. Several such DNA and RNA viruses exist that may be suitable for use in the present invention.

Adenoviral vectors have proven especially useful for gene transfer into eukaryotic cells for vaccine development (Graham F L & Prevec L, In Vaccines: New Approaches to Immunological Problems, Ellis R V ed., 363-390 (Butterworth-Heinemann, Boston, 1992).

“Non-viral” delivery techniques that have been used or proposed for gene therapy include DNA-ligand complexes, adenovirus-ligand-DNA complexes, direct injection of DNA, CaPO₄ precipitation, gene gun techniques, electroporation, and lipofection. Mulligan R C, Science 260: 926-932 (1993). Any of these methods are widely available to one skilled in the art and would be suitable for use in the present invention. Other suitable methods are available to one skilled in the art, and it is to be understood that the present invention may be accomplished using any of the available methods of transfection. Lipofection maybe accomplished by encapsulating an isolated DNA molecule within a liposomal particle and contacting the liposomal particle with the cell membrane of the target cell. Liposomes are self-assembling, colloidal particles in which a lipid bilayer, composed of amphiphilic molecules such as phosphatidyl serine or phosphatidyl choline, encapsulates a portion of the surrounding media such that the lipid bilayer surrounds a hydrophilic interior. Unilammellar or multilammellar liposomes can be constructed such that the interior contains a desired chemical, drug, or, as in the instant invention, an isolated DNA molecule. Delivery by transfection, by liposome injections, or by polycationic amino polymers may be achieved using methods which are well known in the art (see, e.g., Goldman, C. K. et al., Nature Biotechnology 15:462-466 (1997)).

Two types of modified solid tumor stem cells of particular interest are deletion mutants and over-expression mutants. Deletion mutants are wild-type cells that have been modified genetically so that a single gene, usually a protein-coding gene, is substantially deleted. Deletion mutants also include mutants in which a gene has been disrupted so that usually no detectable mRNA or bioactive protein is expressed from the gene, even though some portion of the genetic material may be present. In addition, in some embodiments, mutants with a deletion or mutation that removes or inactivates one activity of a protein (often corresponding to a protein domain) that has two or more activities, are used and are encompassed in the term “deletion mutants.” Over-expression mutants are wild-type cells that are modified genetically so that at least one gene, most often only one, in the modified solid tumor stem cell is expressed at a higher level as compared to a cell in which the gene is not modified.

Genetically modified solid tumor stem cells can be subjected to tissue culture protocols known in the art (see, U.S. Pat. Nos. 5,750,376 and 5,851,832, Spector et al., Cells. A Laboratory Manual (Cold Spring Harbor Press, Cold Spring Harbor, N.Y., 1998)). Tumor stem cells can be genetically modified in culture to promote differentiation, cell death, or immunogenicity. For example, tumor stem cells can be modified to enhance expression of products that direct an immune response against the patient's solid tumor. Alternatively, the solid tumor stem cells can be subjected to various proliferation protocols in vitro prior to genetic modification. The protocol used depends upon the type of genetically modified solid tumor stem cell or solid tumor stem cell progeny desired. Once the cells have been subjected to the differentiation protocol, they are again assayed for expression of the desired protein. Cells having the desired phenotype can be isolated and implanted into recipients in need of the protein or biologically active molecule that is expressed by the genetically modified cell. Such molecules can enhance tumor regression or inhibit tumor spread.

In vitro models of solid tumor development, in vivo models, and methods for screening effects of drugs on solid tumor stem cells. Solid tumor stem cells and solid tumor stem cell progeny cultured in vitro or in vivo (in the xenograft model described in U.S. Pat. Appl. Pub. No. 2002/0119565) can be used for the screening of potential therapeutic compositions. These compositions for the treatment of solid tumors can be applied to these cells in culture at varying dosages, and the response of these cells monitored for various time periods. Physical characteristics of these cells can be analyzed by observing cells by microscopy. The induction of expression of new or increased levels of proteins such as enzymes, receptors and other cell surface molecules can be analyzed with any technique known in the art see, Clarke et al., Proc. Natl. Acad. Sci. USA 92: 11024-11028 (1995), which can identify the alteration of the level of such molecules. These techniques include immunohistochemistry, using antibodies against such molecules, or biochemical analysis. Such biochemical analysis includes protein assays, enzymatic assays, receptor binding assays, enzyme-linked immunosorbant assays (ELISA), electrophoretic analysis, analysis with high performance liquid chromatography (HPLC), Western blots, and radioimmune assays (RIA). Nucleic acid analysis such as Northern blots can be used to examine the levels of mRNA coding for these molecules or PCR.

In some embodiments, the identification of the tumorigenic cell is used in selecting a treatment course of action for a subject. For example, in some embodiments, the treatment course of action comprises administration of a Notch 4 pathway inhibitor to the subject. In other embodiments, the treatment course of action comprises administration of a drug that initiates mitochondrial apoptosis (e.g., regulators of Bak and Bax, regulators of Bcl-2 and BCl_{XL}, regulators of electron transfer—see e.g., U.S. patent application. No. 20030119029, herein incorporated by reference). In some embodiments, the treatment course of action comprises administration of a γ-secretase inhibitor to the subject. γ-secretase inhibitors include, but are not limited to, those described in U.S. Appl. Pub. Nos 20030216380, 20030135044, 20030114387, 20030100512, 20030055005, 20020013315 and U.S. Pat. No. 6,448,229, each of which is herein incorporated by reference, as well as commercially available inhibitors (e.g., from Calbiochem). In some embodiments, the treatment course of action comprises administration of a Manic Fringe inhibitor to the subject. Manic Fringe inhibitors include, but are not limited to, anti-Manic Fringe antibodies, siRNA molecules targeted at Manic Fringe expression, small molecules that inhibit Manic Fringe and the like.

Cells treated with pharmaceutical compositions can be transplanted into an animal (such as in the xenograft model), and their survival, ability to form tumors, and biochemical and immunological characteristics examined.

Some embodiments of the invention provide the means and methods for classifying tumors based upon the profiling of solid tumor samples by comparing a gene expression signature of a cancer sample to a cancer stem cell gene expression signature. Tumor stem cell gene expression signatures can be used as predictors of distant metastases and death. The microarray data of the invention identifies cancer stem cell markers likely to play a role in breast cancer development, progression, and/or maintenance, while also identifying cancer stem cell gene signatures useful in classifying breast tumors into low and high risk of, for example, metastasis and death. Classification based on the detection of differentially expressed polynucleotides and/or proteins that comprise a cancer gene profile when compared to a cancer stem cell gene signature can be used to predict clinical course, predict sensitivity to chemotherapeutic agents, guide selection of appropriate therapy, and monitor treatment response. Furthermore, following the development of therapeutics targeting such cancer stem cell markers, detection of cancer gene signatures described in detail below will allow the identification of patients likely to benefit from such therapeutics.

As described herein, the invention employs methods for clustering genes into gene expression profiles by determining their expression levels in two different cell or tissue samples. The invention further envisions using these gene profiles as compared to a cancer stem cell gene signature to predict clinical outcome including, for example, metastasis and death. The microarray data of the present invention identifies gene profiles comprising similarly and differentially expressed genes contained on the Affymetrix HG-U133 array between two tissue samples including between tumor stem cells and normal breast epithelium, non-tumorigenic tumor cells and normal breast epithelium, and tumor stem cells and non-tumorigenic tumor cells (U.S. Prov. Appl. No. 60/690,003). These broad gene expression profiles can then be further refined, filtered, and subdivided into gene signatures based on various different criteria including, but not limited to, fold expression change, statistical analyses (e.g. t-test P value from multiple compared samples), biological function (e.g., cell cycle regulators, transcription factors, proteases, etc.), some therapeutic targets (e.g., genes encoding extracellular membrane associated proteins suitable for antibody based therapeutics), identified expression in additional patient samples, and ability to predict clinical outcome.

Thus in some embodiments of the invention the genes differentially expressed in tumor stem cells versus normal breast epithelium are subdivided into different cancer stem cell gene signatures based on their fold expression change. For example genes with 2 to 2.5 fold elevated (or reduced, or both elevated and reduced) expression in tumor stem cells can comprise one tumor stem cell gene signature, genes with 2.5 to 3 fold elevated (or reduced, or both) expression can comprise another tumor stem cell gene signature. Alternatively, all genes above a certain fold expression change are included in a tumor stem cell gene signature. For example, all genes with a 1 fold or more reduced (or elevated, or both) expression in tumor stem cells can comprise one tumor stem cell gene signature, all genes with a 2 fold or more reduced (or elevated, or both) expression in tumor stem cells can comprise another tumor stem cell gene signature, and so on. In other embodiments, the genes differentially expressed in tumor stem cells versus normal breast epithelium are filtered by using statistical analysis. For example, all genes with elevated (or reduced, or both) expression with a t-test P value across samples from 0.01 and 0.005 can comprise one tumor stem cell gene signature, all genes with elevated (or reduced, or both) expression with a t-test P value across samples of 0.005 and 0.001 can comprise another tumor stem cell gene signature, and so on. Furthermore, gene expression analysis of independent patient samples or different cell lines can be compared to any cancer stem cell gene signature generated as described above. A tumor stem cell gene signature can be modified, for example, by calculating individual phenotype association indices as described (Glinsky et al., Clin. Cancer Res. 10:2272 (2004)) to increase or maintain the predictive power of a given tumor stem cell gene signature. In addition a tumor stem cell gene signature can be further narrowed or expanded gene by gene by excluding or including genes subjectively (e.g. inclusion of a some therapeutic target or exclusion of a gene included in another gene signatures).

In yet further embodiments, a broad gene expression profile such as those generated by the Affymetrix HG-U133 array analyses of the present invention can be further refined, filtered, or subdivided into gene signatures based on two or more different criteria. In some embodiments of the present invention the genes differentially expressed in tumor stem cells versus normal breast epithelium are subdivided into different tumor stem cell gene signatures based on their fold expression change as well as their biological function. For example, all genes involved in cell cycle regulation with 3 to 3.5 fold elevated (or reduced, or both) expression in tumor stem cells versus normal breast epithelium can comprise one tumor stem cell gene signature, all genes involved in cell cycle regulation with 3.5 to 4 fold elevated (or reduced, or both) expression can comprise another tumor stem cell gene signature, all genes encoding extracellular membrane associated proteins with 4 fold or more elevated (or reduced, or both) expression can comprise another tumor stem cell gene signature, all genes encoding extracellular membrane associated proteins with 5 fold or more elevated (or reduced, or both) expression can comprise yet another tumor stem cell gene signature.

In some embodiments, the genes differentially expressed in tumor stem cells versus normal breast epithelium are divided into different tumor stem cell gene signatures based on their fold expression change and by statistical analysis. The microarray analysis of the invention was used to identify genes with two-fold reduced and two-fold elevated expression in tumorigenic cells versus normal breast epithelium. This tumor stem cell gene signature was then further filtered by the P value of a t-test between the tumorigenic and normal breast epithelium samples to generate the 186 cancer stem cell gene signature comprising an increasingly restricted number of genes (see Table 1).

TABLE 1
186 Cancer Stem Cell Gene Signature

EMP1, CHPT1, TPD52, ERBB4, PRSS16, SH3BGRL,
KIAA1287, ARPC5, SLC25A25, DHRS4, ELP4, C21orf86,
LOC286505, COPB2, C9orf64, FLJ13456, EIF4E2, MAPK14,
TMC4, ZDHHC2, FLJ10587, CEBPD, KIAA1217, MMP7,
CSTF1, KIAA1600, DHRS6, VIL2, MGC45840, NPD014, AIM1,
AMMECR1, CG018, PILRB, PDE8A, TOB2, RAB23, FLJ10774,
LOC439994, ETAA16, DNAJB1, CD59, KIAA0792, NSF,
C7orf2, C11orf17, C7orf25, MR-1, SFPQ, HSPA2, LDHA,
DKFZP566D1346, JTV1, HNMT, STAM, LOC130576,
SERTAD1, LOC255783, FLJ31795, DKFZP564I0422, NOL8,
ECHDC2, CIRBP, SCGN, GOLGIN-67, KLHL20, 8-Mar,
GTPBP1, THUMPD3, AFURS1, KIAA0276, CITED4, SGKL,
C10orf9, C6orf107, C4orf7, PLP2, FLJ90709, FLJ11752, ATXN3,
ICMT, CXCL2, NGFRAP1L1, RAD23B, CNOT4, DNMT3A,
FAM53C, C5orf18, NUDT5, FLJ12439, GTF3C3, RNF8, THEM2,
FLNB, STC2, KIAA0052, DNAPTP6, GABARAPL1, MGP,
DKFZP586A0522, ALG2, SWAP70, FLJ39370, ELL2, GNPDA1,
CDW92, DCBLD1, TUBB, HS2ST1, CAP350, TICAM2,
KIAA0146, BCL2, ISGF3G, MLF1, ETNK1, KLF10, NUP37,
DBR1, METTL2, C10orf7, LARS, B7-H4, SNX6, MAST4,
MGC45564, SRP54, LOC80298, DKFZP564D172, HAN11,
ERN1, FLJ20530, ATIC, NCE2, HSPC163, APLP2, CASP8,
GAPD, NUCKS, SNRPN, PBP, KDELR3, ARGBP2, LRP2, LTF,
C16orf33, LOC283481, ETS1, IER5, CSNK2A1, LOC388279,
PLAA, GSK3B, LRPAP1, MAFF, MGC4399, DUSP10, SCNM1,
PSMA5, NEBL, NDEL1, AGPS, PNAS-4, ZBTB20, CLTC,
DPF2, MAPT, MGC15429, ALKBH, PAK2, WFDC2, STK39,
WEE1, DNAJC13, SSR1, DKFZP564K0822, KIAA0436,
MGC4251, LOC144233, IRX3, C7orf36, FLJ12806, PGK1,
CYP4V2, FLJ37953, XPR1

The invention further embodies the use of these tumor stem cell gene signatures to predict clinical outcome including, but not limited to, metastasis and death. Any independent patient population that includes gene expression analysis (e.g microarray analysis, immunohistochemical analysis, etc) or tumor samples suitable for gene expression analysis (e.g. frozen tissue biopsies, paraffin embedded tumor tissue samples, etc) along with determined clinical parameters or ongoing monitoring of clinical parameters including, for example, lymph node status, metastasis, death, etc. can be used to assess the ability of a tumor stem cell gene signature to predict clinical outcomes. Many statistical analyses can be used to determine predictive ability. These include, for example, Kaplan-Meier survival analysis, Cox proportional hazard survival analysis, chi- square analysis, or multivariate analysis.

The invention therefore establishes the 186 cancer stem cell gene signature as a predictor of poor clinical outcome. In some embodiments of the present invention this cancer stem cell gene signature is used clinically to classify tumors as low or high risk and to assign a tumor to a low or high-risk category. The cancer stem cell gene signature can further be used to provide a diagnosis, prognosis, and/or select a therapy based on the classification of a tumor as low or high risk as well as to monitor a diagnosis, prognosis, and/or therapy over time. If it is known that a patient has a tumor that expresses the genes comprising a cancer stem cell gene signature and thus has a poor prognosis, a more aggressive approach to therapy can be warranted than in tumors not falling within these subcategories. For example, in patients where there is no evidence of disease in lymph nodes (node-negative patients), a decision must be made regarding whether to administer chemotherapy (adjuvant therapy) following surgical removal of the tumor. While some patients are likely to benefit from such treatment, it has significant side effects and is preferably avoided by patients with low risk tumors. Presently it is difficult or impossible to predict which patients would benefit. Knowing that a patient falls into a poor prognosis category can help in this decision. Furthermore, detecting expression of a cancer gene profile that is highly correlated with a cancer stem cell gene signature of the present invention can provide information related to tumor progression. It is well known that as tumors progress, their phenotypic characteristics can change. The invention thus contemplates the possibility that breast tumors can evolve from expressing a cancer gene profile that is highly correlated with a cancer stem cell gene signature to not (or vice versa) either in response to therapy or in response to lack of therapy. Thus detection of a cancer gene profile that either correlates with or fails to correlate with a cancer stem cell gene signature can be used to detect such progression and alter therapy accordingly.

It is well known in the art that some tumors respond to certain therapies while others do not. At present, there is very little information that can be used to determine, prior to treatment, the likelihood that a specific tumor will respond to a given therapeutic agent. Many compounds have been tested for anti-tumor activity and appear to be effective in only a small percentage of tumors. Due to the current inability to predict which tumors will respond to a given agent, these compounds have not been developed as therapeutics. This problem reflects the fact that current methods of classifying tumors are limited. However, the present invention offers the possibility of identifying tumor subgroups and characterizing tumors by a significant likelihood of response to a given agent. Tumor sample archives containing tissue samples obtained from patients that have undergone therapy with various agents are available along with information regarding the results of such therapy. In general such archives consist of tumor samples embedded in paraffin blocks. These tumor samples can be analyzed for their expression of polypeptides that are then compared to the polypeptides encoded by the genes comprising the cancer stem cell signature of the present invention. For example, immunohistochemistry can be performed using antibodies that bind to the polypeptides. Alternatively these tumor samples can be analyzed by their expression of polynucleotides that are then compared to the polynucleotides encoded by the genes comprising a cancer stem cell signature of the present invention. For example, RNA can be extracted from the tumor sample and RT-PCR used to quantitatively amplify mRNAs that would then be compared to the mRNAs comprising a cancer stem cell signature. Tumors belonging to one or more of thirteen cancer stem cell subclasses can be identified on the basis of this information. It is then possible to correlate the expression of the cancer gene profile with a cancer stem cell gene signature predicted response of the tumor to therapy, thereby identifying particular compounds that show a superior efficacy against tumors of a certain subclass as compared with their efficacy against tumors overall or against tumors not falling within the subclass. Once such compounds are identified it will be possible to select patients whose tumors fall into a particular subclass for additional clinical trials using these compounds. Such clinical trials, performed on a selected group of patients, are more likely to demonstrate efficacy. The reagents provided herein, therefore, are valuable both for retrospective and prospective trials.

In the case of prospective trials, detection of expression of one or more of the genes or encoded proteins in a cancer gene profile that correlates with a cancer stem cell signature can be used to stratify patients prior to their entry into the trial or while they are enrolled in the trial. In clinical research, stratification is the process or result of describing or separating a patient population into more homogeneous subpopulations according to specified criteria. Stratifying patients initially rather than after the trial is frequently some (including by regulatory agencies such as the U.S. Food and Drug Administration involved in the approval process for a medication), and stratification is frequently useful in performing statistical analysis of the results of a trial. In some cases stratification can be required by the study design. Various stratification criteria can be employed in conjunction with detection of expression of one or more cancer gene profiles that correlate with a cancer stem cell gene signature. Commonly used criteria include age, family history, lymph node status, tumor size, tumor grade, etc. Other criteria that can be used include, but are not limited to, tumor aggressiveness, prior therapy received by the patient, estrogen receptor (ER) and/or progesterone receptor (PR) positivity, Her2/neu status, p53 status, etc. Ultimately, once compounds that exhibit superior efficacy against cancer gene profile tumors that are highly correlated with cancer stem cell gene signature are identified, reagents for detecting expression of the gene profile can be used to guide the selection of appropriate therapy for additional patients. Thus, by providing reagents and methods for classifying tumors based on their expression of a cancer gene profile that is compared to a cancer stem cell gene signature, the present invention provides a means to identify a patient population that can benefit from potentially promising therapies that have been abandoned due to inability to benefit broader or more heterogeneous patient populations and further offers a means to individualize cancer therapy.

Information regarding the expression of cancer stem cell signature genes is thus useful even in the absence of specific information regarding their biological function or role in tumor development, progression, and maintenance. Although the reagents disclosed herein find particular application with respect to breast cancer, the invention also contemplates their use to provide diagnostic and/or prognostic information for other cancer types including but not limited to: biliary tract cancer; bladder cancer; brain cancer including glioblastomas and medulloblastomas; cervical cancer; choriocarcinoma; colon cancer; ondometial cancer; esophageal cancer; gastric cancer; hematological neoplasms including acute lymphocytic and myelogenous leukemia; multiple myeloma; AIDS- associated leukemnius and adult T-cell leukemia lymphoma; intraepithelial neoplasms including Bowen's disease and Paget's disease; liver cancer; lung cancer; lymphomas including Hodgkin's disease and lymnphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and modullar carcinoma; and renal cancer including adenocarcinoma and Wilms tumor.

In some embodiments of the invention, the cancer stem cell signature is used experimentally to test and assess lead compounds including, for example, small molecules, siRNAs, and antibodies for the treatment of cancer. For example, tumor cells from a patient can be screened for expression of a cancer stem cell gene signature and then transplanted into the xenograft model described herein and the effect of test compounds, such as for example antibodies against one or more cancer stem cell markers described herein, tested for effects on tumor growth and survival. Furthermore a cancer gene profile can be determined following treatment and the cancer gene profile compared to a cancer stem cell gene signature to assess the effectiveness of the therapy and in turn guide a future treatment regimen. In addition the efficacy of test compounds can be assessed against different tumor subclasses. For example test compounds can be used in xenografts of tumors that express a cancer gene profile that is highly correlated with a tumor stem cell gene signature versus tumors having a gene profile that does not correlate with the tumor stem cell gene signature or that express other gene signature such as, for example, a serum response gene signature (Chang et al., 2005, PNAS 102:3738). Any differences in response of the different tumor subclasses to the test compound are determined and used to optimize treatment for particular classes of tumors.

The cancer stem cell gene signature was previously identified from genes that are expressed at decreased or elevated levels in tumor stem cells compared to normal breast epithelium. Thus in certain embodiments of the invention expression levels of mRNA, or amplified or cloned version thereof, are determined from a tumor sample by hybridization to polynucleotides that represent each particular gene comprising a cancer stem cell gene signature. some polynucleotides of this type contain at least about 20 to at least about 32 consecutive base pairs of a gene sequence that is not found in other gene sequences. Even more some are polynucleotides of at least or about 50 to at least or about 400 base pairs of a gene sequence that is not found in other gene sequences. Such polynucleotides are also referred to as polynucleotide probes in that they are capable of hybridizing to sequences of the genes, or unique portions thereof, described herein. The sequences can be those of mRNA encoded by the genes, the corresponding cDNA to such mRNAs, and/or amplified versions of such sequences. In one some embodiments of the invention a cancer gene profile is detected by polynucleotide probes that comprise the polynucleotides comprising the stem cell gene signature immobilized on an array (such as a cDNA microarray).

In some embodiments of the invention, all or part of the disclosed polynucleotides of a cancer stem cell gene signature are amplified and detected by methods such as the polymerase chain reaction (PCR) and variations thereof, such as, but not limited to, quantitative PCR (Q-PCR), reverse transcription PCR (RT-PCR), and real-time PCR (including means of measuring the initial amounts of mRNA copies for each sequence in a sample). Real-time RT-PCR or real-time Q-PCR can be used. Such methods utilize one or two primers that are complementary and hybridize to portions of a disclosed sequence, where the primers are used to prime nucleic acid synthesis. The newly synthesized nucleic acids are optionally labeled and can be detected directly or by hybridization to a polynucleotide of the invention. Additional methods to detect expressed nucleic acids include RNAse protection assays, including liquid phase hybridizations, and in situ hybridization of cells or tissue samples.

In yet other embodiments of the invention, gene expression can be determined by analysis of protein expression. Protein expression can be detected by use of one or more antibodies specific for one or more epitopes of individual gene products (proteins), or proteolytic fragments thereof, of a cancer stem cell signature in a tumor sample. Detection methodologies suitable for use in the practice of the invention include, but are not limited to, immunohistochemistry of cells in a tumor sample, enzyme linked immunosorbent assays (ELISAs) including antibody sandwich assays of cells in a tumor sample, mass spectroscopy, immuno-PCR, FACS, and protein microarrays.

It is envisioned that any patient sample can be used to detect a cancer stem cell signature. Importantly, though the cancer stem cell signatures were discovered from a comparison of cancer stem cells against a non-tumorigenic tissue, such as for example, normal breast tissue, its prognostic ability was identified from microarray analysis of unfractionated, and thus heterogeneous, breast tumor samples normalized either against a reference set of tumor samples (van't Veer et al., 2002, Nature 415:530; van de Vijver et al., 2002, N. Eng. J. Med. 347:1999) or to a target intensity (Wang et al., 2005, Lancet 365:671). Thus unfractioned tumor samples, including but not limited to a solid tissue biopsy, fine needle aspiration, or pleural effusion, can be used for detecting a cancer stem cell signature in the tumor sample and generating a cancer gene profile. More selective samples that are isolated from a heterogeneous patient sample such as, for example, by isolating tumorigenic cancer cells, laser capture microdissections, etc. can also be used. Alternatively the sample can permit the collection of cancer cells as well as normal cells for analysis so that the gene expression patterns for each sample can be determined and compared to a cancer stem cell gene signature to generate a cancer gene profile.

eDNA Microarray Technology

cDNA microarrays consist of multiple (usually thousands) of different cDNAs spotted (usually using a robotic spotting device) onto known locations on a solid support, such as a glass microscope slide. The cDNAs are typically obtained by PCR amplification of plasmid library inserts using primers complementary to the vector backbone portion of the plasmid or to the gene itself for genes where sequence is known. PCR products suitable for production of microarrays are typically about 0.5 and 2.5 kB in length.- Full length cDNAs, expressed sequence tags (ESTs), or randomly chosen cDNAs from any library of interest can be chosen. ESTs are partially sequenced cDNAs as described, for example, in Hillier, et al., 1996, 6:807-828. Although some ESTs correspond to known genes, frequently very little or no information regarding any particular EST is available except for a small amount of 3′ and/or 5′ sequence and, possibly, the tissue of origin of the mRNA from which the EST was derived. As will be appreciated by one of ordinary skill in the art, in general the cDNAs contain sufficient sequence information to uniquely identify a gene within the human genome. Furthermore, in general the cDNAs are of sufficient length to hybridize, such as selectively, specifically or uniquely, to cDNA obtained from mRNA derived from a single gene under the hybridization conditions of the experiment.

In a typical microarray experiment, a microarray is hybridized with differentially labeled RNA, DNA, or cDNA populations derived from two different samples. Most commonly RNA (either total RNA or poly A⁺ RNA) is isolated from cells or tissues of interest and is reverse transcribed to yield cDNA. Labeling is usually performed during reverse transcription by incorporating a labeled nucleotide in the reaction mixture. Although various labels can be used, most commonly the nucleotide is conjugated with the fluorescent dyes Cy3 or Cy5. For example, Cy5-dUTP and Cy3-dUTP can be used. cDNA derived from one sample (representing, for example, a particular cell type, tissue type or growth condition) is labeled with one fluorophore while cDNA derived from a second sample (representing, for example, a different cell type, tissue type, or growth condition) is labeled with the second fluorophore. Similar amounts of labeled material from the two samples are cohybridized to the microarray. In the case of a microarray experiment in which the samples are labeled with Cy5 (which fluoresces red) and Cy3 (which fluoresces green), the primary data (obtained by scanning the microarray using a detector capable of quantitatively detecting fluorescence intensity) are ratios of fluorescence intensity (red/green, R/G). These ratios represent the relative concentrations of cDNA molecules that hybridized to the cDNAs represented on the microarray and thus reflect the relative expression levels of the mRNA corresponding to each cDNA/gene represented on the microarray.

Each microarray experiment can provide tens of thousands of data points, each representing the relative expression of a particular gene in the two samples. Appropriate organization and analysis of the data is of key importance, and various computer programs that incorporate standard statistical tools have been developed to facilitate data analysis. One basis for organizing gene expression data is to group genes with similar expression patterns together into clusters. A method for performing hierarchical cluster analysis and display of data derived from microarray experiments is described in Eisen et al., Proc Natl Acad Sci USA 95:14863-14868 (1998). As described therein, clustering can be combined with a graphical representation of the primary data in which each data point is represented with a color that quantitatively and qualitatively represents that data point. By converting the data from a large table of numbers into a visual format, this process facilitates an intuitive analysis of the data. Additional information and details regarding the mathematical tools and/or the clustering approach itself can be found, for example, in Sokal & Sneath, Principles of numerical taxonomy, xvi, 359, W. H. Freeman, San Francisco, 1963; Hartigan, Clustering algorithms, xiii, 351, Wiley, New York, 1975; Paull et al., J. Natl. Cancer Inst. 81:1088-92 (1989); Weinstein et al., Science 258:447-51 1992); van Osdol et al., J. Natl. Cancer Inst. 86:1853-9 (1994); and Weinstein et al., Science, 275:343-9 (1997).

Additional information describing methods for fabricating and using microarrays is found in U.S. Pat. No. 5,807,522, which is herein incorporated by reference. Instructions for constructing microarray hardware (e.g., arrayers and scanners) using commercially available parts can be found at http://cmgm.stanford.edu/pbr-own/ and in Cheung et al., 1999, Nat. Genet. Supplement 21:15-19, which are herein incorporated by reference. Additional discussions of microarray technology and protocols for preparing samples and performing microrarray experiments are found in, for example, DNA arrays for analysis of gene expression, Methods Enzymol, 303:179-205, 1999; Fluorescence-based expression monitoring using microarrays, Methods Enzymol, 306: 3-18, 1999; and M. Schena (ed.), DNA Microarrays: A Practical Approach, Oxford University Press, Oxford, UK, 1999.

Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject can visit a medical center to have the sample obtained and sent to the profiling center, or subjects can collect the sample themselves and directly send it to a profiling center. Where the sample comprises previously determined biological information, the information can be directly sent to the profiling service by the subject (e.g., an information card containing the information can be scanned by a computer and the data transmitted to a computer of the profiling center using an electronic communication system). Once received by the profiling service, the sample is processed and a profile is produced (e.g., expression data), specific for the diagnostic or prognostic information desired for the subject.

The profile data is then prepared in a format suitable for interpretation by a treating clinician. For example, rather than providing raw expression data (e.g. examining a number of the markers described in Tables 4-9 as well as Tables A-N), the prepared format can represent a diagnosis or risk assessment for the subject, along with recommendations for particular treatment options. The data can be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject can chose further intervention or counseling based on the results. In some embodiments, the data is used for research use.

For example, the data can be used to further optimize the inclusion or elimination of markers as useful indicators of a particular condition or stage of disease.

Kits

In yet other embodiments, the present invention provides kits for the detection and characterization of cancer (e.g. for detecting one or more of the markers shown in Table 1, or for modulating the activity of a peptide expressed by one or more of markers shown in Table 1). In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In some embodiments, the kits contain all of the components necessary and/or sufficient to perform a detection assay, including all controls, directions for performing assays, and any necessary software for analysis and presentation of results.

Another aspect of the present invention comprises a kit to test for the presence of the polynucleotides or proteins, e.g. in a tissue sample or in a body fluid, of a cancer stem cell signature. The kit can comprise, for example, an antibody for detection of a polypeptide or a probe for detection of a polynucleotide. In addition, the kit can comprise a reference or control sample; instructions for processing samples, performing the test and interpreting the results; and buffers and other reagents necessary for performing the test. In certain embodiments the kit comprises a panel of antibodies for detecting expression of one or more of the proteins encoded by the genes of a cancer stem cell signature. In other embodiments the kit comprises pairs of primers for detecting expression of one or more of the genes of the cancer stem cell signature. In yet other embodiments the kit comprises a cDNA or oligonucleotide array for detecting expression of one or more of the genes of a cancer stem cell signature.

Drug Screening

In some embodiments, the present invention provides drug screening assays (e.g., to screen for anticancer drugs). The screening methods of the present invention utilize stem cell cancer markers identified using the methods of the present invention (e.g., including but not limited to, the stem cell cancer markers shown in Table 1). The screening methods are described in detail in related U.S. Appl. Nos. 60/690,003 and Ser. No. 09/920,517, which are herein incorporated by reference). For example, in some embodiments, the present invention provides methods of screening for compound that alter (e.g., increase or decrease) the expression of stem cell cancer marker genes. In some embodiments, candidate compounds are antisense agents or siRNA agents (e.g., oligonucleotides) directed against cancer markers. In other embodiments, candidate compounds are antibodies that specifically bind to a stem cell cancer marker of the present invention. In certain embodiments, libraries of compounds of small molecules are screened using the methods described herein.

In one screening method, candidate compounds are evaluated for their ability to alter stem cell cancer marker expression by contacting a compound with a cell expressing a stem cell cancer marker and then assaying for the effect of the candidate compounds on expression. In some embodiments, the effect of candidate compounds on expression of a cancer marker gene is assayed by detecting the level of cancer marker mRNA expressed by the cell. mRNA expression can be detected by any suitable method. In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein. In some embodiments, other changes in cell biology (e.g., apoptosis) are detected.

Specifically, the present invention provides screening methods for identifying modulators, i.e., candidate or test compounds or agents (e.g., proteins, peptides, peptidomimetics, peptoids, small molecules or other drugs) which bind to, or alter the signaling or function associated with the cancer markers of the present invention, have an inhibitory (or stimulatory) effect on, for example, stem cell cancer marker expression or cancer markers activity, or have a stimulatory or inhibitory effect on, for example, the expression or activity of a cancer marker substrate. Compounds thus identified can be used to modulate the activity of target gene products (e.g., stem cell cancer marker genes) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly metastatic cancer or eliminating or controlling tumor stem cells to prevent or reduce the risk of cancer.

In some embodiments, the invention provides assays for screening candidate or test compounds that are substrates of a cancer markers protein or polypeptide or a biologically active portion thereof. In another embodiment, the invention provides assays for screening candidate or test compounds that bind to or modulate the activity of a cancer marker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are some for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop etal., J. Med. Chem. 37:1233 (1994).

Libraries of compounds can be presented in solution (e.g., Houghten, Biotechniques 13:412-421 (1992)), or on beads (Lam, Nature 354:82-84 (1991)), chips (Fodor, Nature 364:555-556 (1993)), bacteria or spores (U.S. Pat. No. 5,223,409; herein incorporated by reference), plasmids (Cull et al., Proc. Nad. Acad. Sci. USA 89:18651869 (1992)) or on phage (Scott and Smith, Science 249:386-390 (1990); Devlin Science 249:404-406 (1990); Cwiria et al.,. Proc. Natl. Acad. Sci. 87:6378-6382 (1990); Felici, J. Mol. Biol. 222:301 (1991)).

In some embodiments, an assay is a cell-based assay in which a cell that expresses a stem cell cancer marker protein or biologically active portion thereof is contacted with a test compound, and the ability of the test compound to the modulate cancer marker's activity is determined. Determining the ability of the test compound to modulate stem cell cancer marker activity can be accomplished by monitoring, for example, changes in enzymatic activity. The cell, for example, can be of mammalian origin.

The ability of the test compound to modulate cancer marker binding to a compound, e.g., a stem cell cancer marker substrate, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

In yet other embodiments, a cell-free assay is provided in which a cancer marker protein or biologically active portion thereof is contacted with a test compound and the ability of the test compound to bind to the stem cell cancer marker protein or biologically active portion thereof is evaluated. Some biologically active portions of the cancer markers proteins to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

In some embodiments, the target gene product or the test substance is anchored onto a solid phase. The target gene product/test compound complexes anchored on the solid phase can be detected at the end of the reaction. The target gene product can be anchored onto a solid surface, and the test compound, (which is not anchored), can be labeled, either directly or indirectly, with detectable labels discussed herein.

It can be desirable to immobilize stem cell cancer markers, an anti-cancer marker antibody or its target molecule to facilitate separation of complexed from non-complexed forms of one or both of the proteins, as well as to accommodate automation of the assay. Binding of a test compound to a stem cell cancer marker protein, or interaction of a cancer marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-cancer marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or cancer marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer markers binding or activity determined using standard techniques. Other techniques for immobilizing either cancer markers protein or a target molecule on matrices include using conjugation of biotin and streptavidin. Biotinylated cancer marker protein or target molecules can be prepared from biotin-NHS (N-hydroxy-succinimide) using techniques known in the art (e.g., biotinylation kit, Pierce Chemicals, Rockford, EL), and immobilized in the wells of streptavidin-coated 96 well plates (Pierce Chemical).

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit 11:141-8 [1998]; Hageand Tweed J. Chromatogr. Biomed. Sci. Appl 699:499-525 [1997]). Further, fluorescence energy transfer can also be conveniently utilized, as described herein, to detect binding without further purification of the complex from solution.

The assay can include contacting the stem cell cancer markers protein or biologically active portion thereof with a known compound that binds the cancer marker to form an assay mixture, contacting the assay mixture with a test compound, and determining the ability of the test compound to interact with a cancer marker protein, wherein determining the ability of the test compound to interact with a cancer marker protein includes determining the ability of the test compound to preferentially bind to cancer markers or biologically active portion thereof, or to modulate the activity of a target molecule, as compared to the known compound.

To the extent that stem cell cancer markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, cancer markers protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent WO 94/10300; each of which is herein incorporated by reference), to identify other proteins, that bind to or interact with cancer markers (“cancer marker-binding proteins” or “cancer marker-bp”) and are involved in cancer marker activity. Such cancer marker-bps can be activators or inhibitors of signals by the cancer marker proteins or targets as, for example, downstream elements of a cancer markers-mediated signaling pathway.

Modulators of cancer markers expression can also be identified. For example, a cell or cell free mixture is contacted with a candidate compound and the expression of cancer marker mRNA or protein evaluated relative to the level of expression of stem cell cancer marker mRNA or protein in the absence of the candidate compound. When expression of cancer marker mRNA or protein is greater in the presence of the candidate compound than in its absence, the candidate compound is identified as a stimulator of cancer marker mRNA or protein expression. Alternatively, when expression of cancer marker mRNA or protein is less (i.e., statistically significantly less) in the presence of the candidate compound than in its absence, the candidate compound is identified as an inhibitor of cancer marker mRNA or protein expression. The level of cancer markers mRNA or protein expression can be determined by methods described herein for detecting cancer markers mRNA or protein.

A modulating agent can be identified using a cell-based or a cell free assay, and the ability of the agent to modulate the activity of a cancer markers protein can be confirmed in vivo, e.g., in an animal such as an animal model for a disease (e.g., an animal with prostate cancer or metastatic prostate cancer; or an animal harboring a xenograft of a prostate cancer from an animal (e.g., human) or cells from a cancer resulting from metastasis of a prostate cancer (e.g., to a lymph node, bone, or liver), or cells from a prostate cancer cell line.

This invention further pertains to novel agents identified by the above-described screening assays. Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein (e.g. to treat a human patient who has cancer).

In some embodiments, the present invention provides therapies for cancer (e.g., breast cancer). These therapies include, but are not limited to antisense therapy, genetic therapy, antibody therapy, RNAi therapy and pharmaceutical compositions. Each of these therapies is described in detail in U.S. Appl. Nos. 60/690,003 and Ser. No. 09/920,517. In some embodiments, therapies target cancer markers (e.g., including but not limited to, those shown in Table 1).

### EXAMPLES

### Isolation of Murine Breast Cancer Stem Cells

Tumorigenic Cells in MMTV-Wnt-1 Mice Express CD24 and Thy1

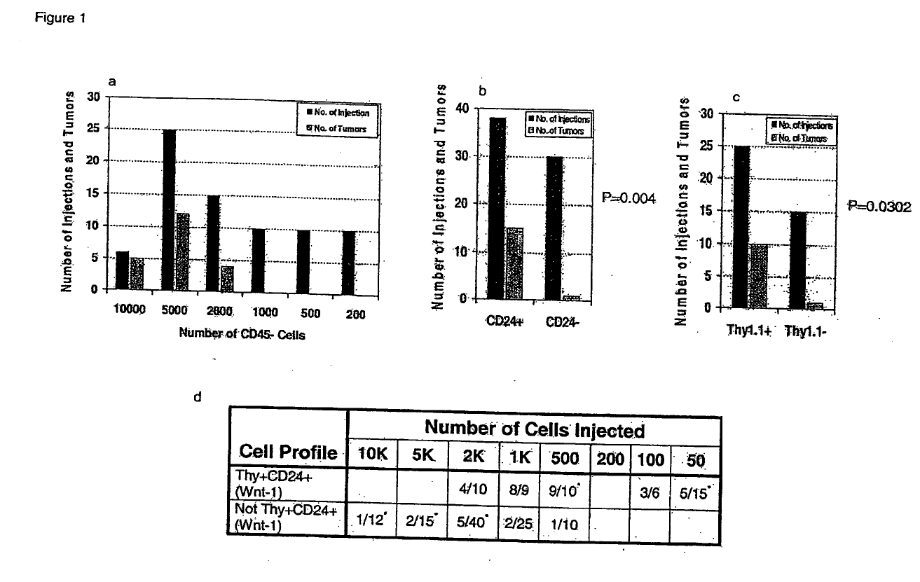

The MMTV-Wnt-1 mouse as a model to study cancer stem cells is described below. The tumorigenicity of various tumor populations was analyzed by flow cytometry using antibodies against select cell surface markers. Viable CD45 negative cells were first tested for tumorigenicity since CD45 is an established exclusion marker for hematopoietic cells. CD45− cells from the Wnt-1 tumors were injected subcutaneously near the upper nipple lines of syngeneic (FVB/NJ) mice in a limiting dilution manner. A cell dose of 1000 cells or less did not produce new tumors (FIG. 1A). At 2000 cell injections, tumors appeared with 15 injections resulting in 4 tumors. By the 10,000 cell doses, a tumor arose from 5 of 6 injections.

Sca-1, Thy-1, and CD24 were tested next as additional markers to help segregate tumorigenic from nontumorigenic populations since these markers had been previously shown to be useful in identifying cells with self-renewing properties 5. These antibodies were used in conjunction with CD45 (to deplete hematopoietic cells) and a viability marker (7-AAD or DAPI to deplete for dead and dying cells). Tumor cells demonstrated differential expression patterns for all three markers, consistent with tumors being composed of heterogeneous population of cells. Sca-1⁺and Sca-1− cells, formed tumors with equal frequency, and therefore, Sca-1 was not useful for enriching a tumor- generating cell population (data not shown). In contrast, CD24 and Thy-1 were both useful markers in differentiating tumorigenic from nontumorigenic populations. When injected into the breast of syngeneic mice, 15 of 38 injections of 1,000 CD24⁺ cells formed tumors , while only 1 of 30 injections of 1,000 CD24− cells did so (p=0.004) (FIG. 1b). Ten of 25 injections of Thy-1⁺ cells resulted in tumors, but only 1 of 15 injections of Thy-1− tumors gave rise to a tumor (p=0.03) (FIG. 1c). This suggested that a tumorigenic population of cells existed within both the CD24⁺ and Thy1⁺ subpopulation of cells.

Thy⁺CD24⁺CD45⁻ Cells Are Enriched for Tumorigenic Cells

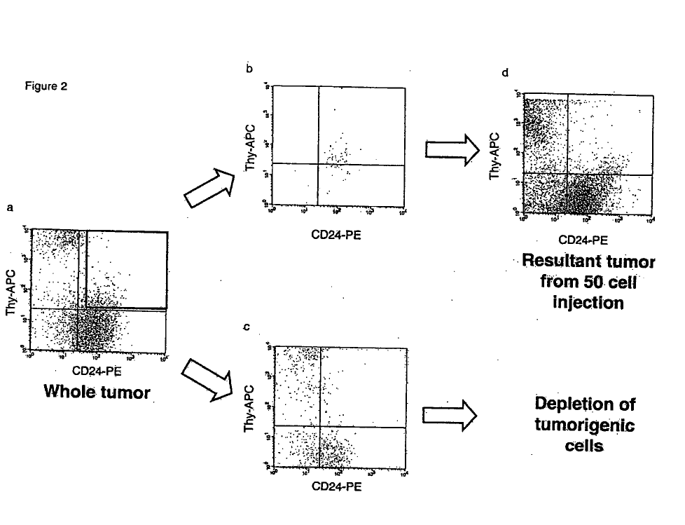

Flow cytometry was then used to isolate cells based on the combination of CD24, Thy-1 and CD45. Approximately 0.5-1% of the total tumor cells (1-3% of the CD45− tumor cells) were Thy-1⁺CD24⁺CD45⁻ (FIG. 2A). Limiting dilutions were done to determine whether the Thy-1⁺CD24⁺CD45⁻ tumor cells were enriched for tumorigenic cells. In 6 of the 7 tumors examined, this was indeed the case. Tumors formed in 8 of 9, 9 of 10, 3 of 6, and 5 of 15 injections of 1,000, 500, 100, and 50 Thy-1⁺CD24⁺CD45⁻ cells, respectively (FIG. 1D). In 6 of 7 tumors tested, the remaining tumor cells that were “Not Thy-1⁺CD24⁺CD45⁻”, were significantly depleted of cells capable of forming tumors. Only 1 of 12 and 2 of 15 injections of 10,000 and 5,000 of the remaining tumor cells formed tumors (FIG. 1D). This data suggests that the tumorigenic cells reside specifically in the small minority of cells which make up the Thy-1⁺CD24⁺ fraction in the tumors.

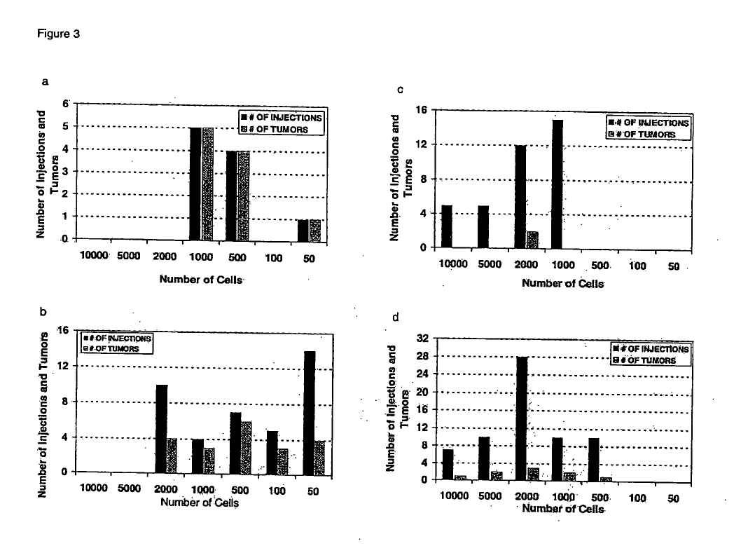

Serial transplantation studies were performed to determine whether Thy-1⁺CD24⁺CD45⁻ cells were able to self-renew. Flow cytometry analysis of secondary tumors generated by as few as 50 Thy-1⁺CD24⁺CD45⁻ cells showed that they contained the same populations of tumorigenic and non-tumorigenic cells as the original tumor (FIGS. 2B and 2D). All 10 injections of 50-1,000 Thy-1⁺CD24⁺CD45⁻ isolated from the primary tumor formed secondary tumors, as opposed to only 2 of 37 injections of 1,000-10,000 of the “Not Thy-1^{+CD}24⁺CD45⁻” tumor cells (FIGS. 3A & 3C). In secondary tumors, 9 of 11 injections of 500-1000 Thy-1⁺CD24⁺CD45⁻ tumor cells gave rise to tertiary tumors, whereas only 3 of 17 injections of 5,000-10,000 of the rest of the CD45⁻ tumor cells did so (FIGS. 3B & 3D). Tertiary tumors, again, contained phenotypically similar cancer cells as those found in de novo tumors. These results demonstrate that tumorigenic cancer cells are enriched in the Thy⁺CD24⁺CD45⁻ cell fraction and are capable of self-renewal to regenerate tumor containing the same heterogeneous population of tumorigenic and non-tumorigenic cells as in the original tumor. The few tumors which grew in the “Not Thy-1⁺CD24⁺CD45⁻” fraction are likely secondary to the inability to completely eliminate Thy-1⁺CD24⁺ cells by sorting. Alternatively, there may be a small population of tumorigenic cells that do not express CD24 or Thy-1.

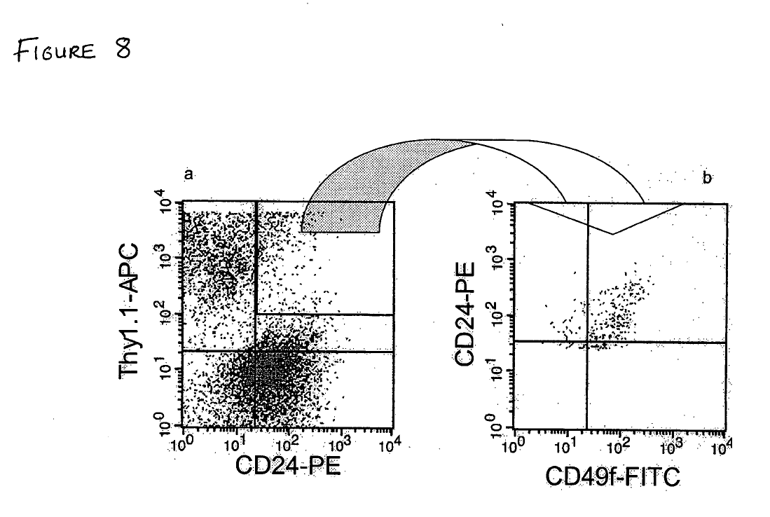

CD49f was also investigated for its use to further enrich for tumorigenic cells. At least 80% of the Thy1⁺CD24⁺CD45⁻ tumorigenic cancer cells were also CD49f⁺ (FIG. 8). Injections at 50 and 500 cell doses of Thy-1⁺CD49f⁺CD24⁺CD45⁻ cells formed tumors, demonstrating that tumorigenic cancer cells express CD49f (FIG. 1D).

Normal Mammary Cells With Regenerative Capacity in vivo are Thy1⁺CD24⁺CD49f⁺CD45−

To help determine the cellular origin of Wnt-1 tumorigenic cells, normal murine mammary cells expressing Thy1⁺CD24⁺CD49f⁺CD45⁻ were analyzed to assess their capability of reconstituting mammary tissue in recipient mice cleared of their mammary fat pads. It was reasoned that Wnt-1 tumorigenic cells are derived from normal cells with similar proliferative and self-renewing capacity. To test this hypothesis, the fact that Thy1 exists as two different alleles, and therefore this marker can be used to differentiate cells from donor and recipient mice was analyzed. Mammary fat pads isolated from donor 3-4 week old C57BL/Kα-1.1/Thy1.1 mice were dissected and dissociated into single-cell suspensions for flow cytometry. All subpopulations of CD45 expressing cells were depleted to remove cells of hematopoietic lineage. To first determine the fewest number of sorted cells optimal for injection, the ability of CD45- cells to form mammary outgrowths in mice cleared of their fourth mammary fat pads was tested. At 6 weeks post-injection, recipient mice injected with CD45⁻ cells showed new mammary outgrowths in the area that was cleared of the original fat pad. Limiting dilutions of CD45⁻ cells were performed to determine the level of enrichment for cells with duct-regenerative capacity (Table 2). The results showed that 7 of 7 injections of 100,000, 6 of 6 injections of 50,000 and 0 of 2 injections of 10,000 cells engrafted into recipient mice and produced ductal outgrowths.

TABLE 2
Thy1.1⁺CD24⁺CD49f⁺CD45− are capable
of producing ductal outgrowths in vivo

| | Number of Cells Injected | | | | | |
|---|---|---|---|---|---|---|
| Cell Profile | 100K | 50K | 10K | 5K | 2K | 1K |
| CD45− | 7/7a | 6/6a | 0/2a | — | — | — |
| Thy1.1⁺CD24⁺CD49f⁺ | — | — | 5/5 | 2/4 | 7/7 | 1/1 |
| Not | | — | 1/5* | 0/5 | — | — |
| Thy1.1⁺CD24⁺CD49f⁺ | | | | | | |

Donor mammary cells were isolated using flow cytometry based on the indicated marker expression and injected into recipient mice. Analysis of recipient mice was performed 6 weeks after the initial cell injection. (a) CD45⁻ limiting dilution transplants were assessed based only on visualization of a new ductal outgrowth into the cleared fat pad region. Transplants of Thy1.1^{+CD}24⁺CD49f⁺CD45⁻ and “Not Thy1.1⁺CD24⁺CD49f⁺CD45⁻” cells were evaluated by determining the contribution of Thy1.1 donor cells by flow cytometry in addition to visualization of new ductal outgrowths of the cleared fat pad region. Thy1.1⁺CD24⁺CD49f⁺CD45⁻ cells consistently produced ductal outgrowths with 10,000, 5,000, and 2,000 cell injections. In contrast, the “Not Thy1.1⁺CD24⁺CD49f⁺CD45⁻” mammary cells were unable to consistently grow new mammary ducts even at injections of 10,000 cells, suggesting that the Thy1.1⁺CD24⁺CD49f⁺CD45⁻ population is enriched for duct-regenerating cells. *The one fat pad which grew from the “Not Thy1.1⁺CD24⁺CD49f⁺CD45⁻” contained a partial ductal outgrowth that was not positive for Thy1.1 donor cells.

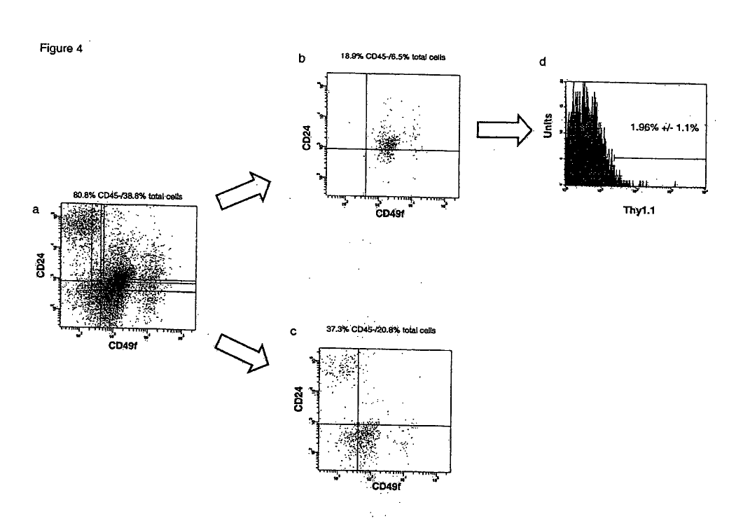

The ability of Thy1 and CD24 to be used to enrich normal mammary duct-regenerating cells was also analyzed. Thy1.1⁺CD24⁺CD49f⁺CD45⁻ and “Not Thy1.1⁺CD24⁺CD49f⁺CD45⁻” cells were isolated by flow cytometry (FIG. 4A-C). Each population was then injected into recipient C57BL/6 mice (which harbor the Thy1.2 allele) that had their fourth mammary fat pads cleared. Contribution of Thy1.1 cells to growth in the region of the cleared fourth fat pads of Thy1.2 recipient mice was assessed by visual confirmation of a ductal outgrowth as well as by flow cytometry analysis of cells isolated from the reconstituted area for Thy1.1 expression. At six weeks post-injection, mice injected with Thy1.1⁺CD24⁺CD49f⁺CD45⁻ cells showed regeneration of mammary tissue in the region of the cleared fourth fat pad whereas mice receiving mock injections did not. In limiting dilution experiments with Thy1⁺CD24⁺CD49f⁺CD45⁻ cells, we found that 5 of 5 injections of 10,000 cells, 2 of 4 injections of 5,000 cells, 7 of 7 injections bf 2000 cells, and 1 of 1 injection of 1000 cells were able to produce ductal outgrowths into the cleared mammary fat pads (Table 1). In addition, these ductal outgrowths contained Thy1.1 donor cells as analyzed by flow cytometry comprising 1.96±1.10% of the total cells analyzed from the cleared area (FIG. 4D). In contrast, injections of “Not Thy1⁺CD24⁺CD49f⁺CD45⁻” cells did not have the same regenerative capacity as Thy1⁺CD24⁺CD49f⁺CD45⁻ cells. Only 1 of 5 injections of 10,000 cells and none of 5 cell injections of 5000 cells of the “Not Thy1⁺CD24⁺CD49f⁺CD45⁻” produced ductal outgrowths (Table 2). Notably, the cells in the single outgrowth in a mouse injected with the “Not Thy1⁺CD24⁺CD49f⁺CD45⁻” cells only expressed Thy-1.2, suggesting that it arose from residual host cells. Based on the results of this limiting dilution analysis, the Thy1⁺CD24⁺CD49f⁺ population of normal mammary cells are 10-50-fold enriched for duct-regenerating cells.

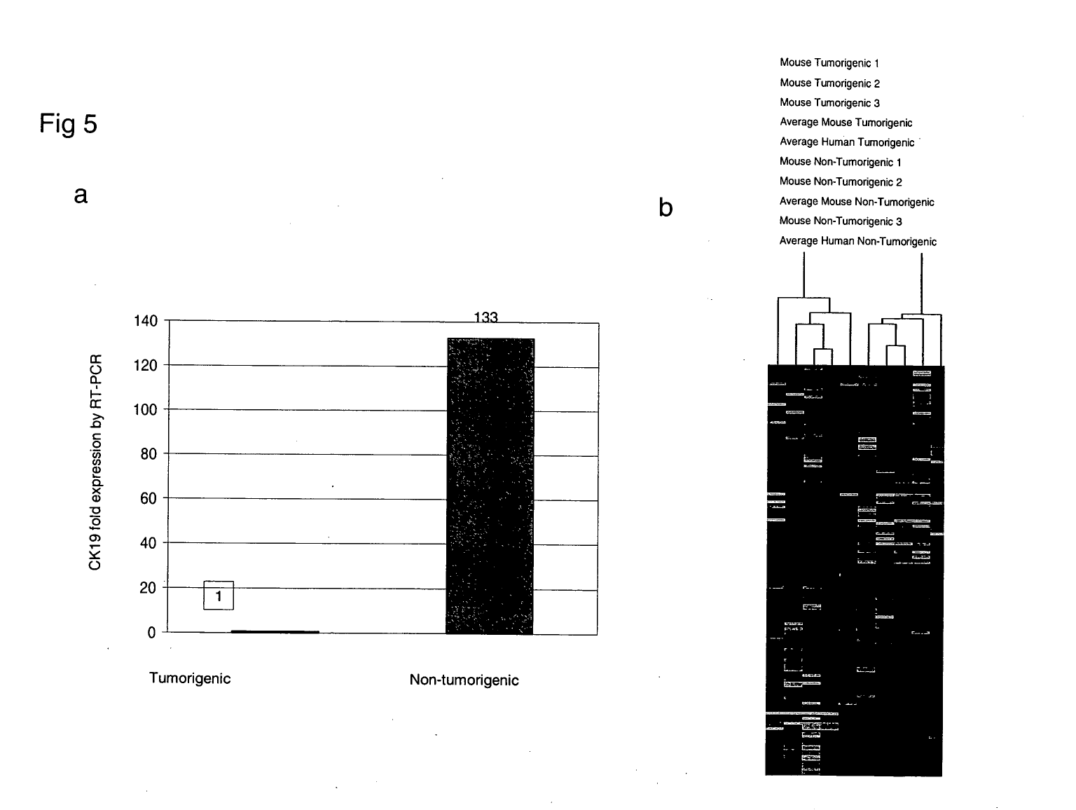

These engraftment studies using normal Thy1⁺CD24⁺CD49f⁺ mammary cells demonstrate that marker expression is indeed conserved between the MMTV- Wnt-1 tumor-regenerating population and the duct-regenerating cells of the normal mammary gland. Furthermore, the “Not Thy1⁺CD24⁺CD49f⁺CD45⁻” Wnt-1 tumor population expressed more than 100 fold higher levels of cytokeratin 19, a differentiation marker for breast epithelial cellsl9 by real-time PCR (FIG. 5A). These results represent the first in vivo study using phenotypic marker expression to identify analogous tumorigenic and normal mammary populations that are enriched for self-renewing populations of malignant and normal cells, respectively.

Thy1 Marks Only Clusters of Neoplastic Cells in Tumor Sections

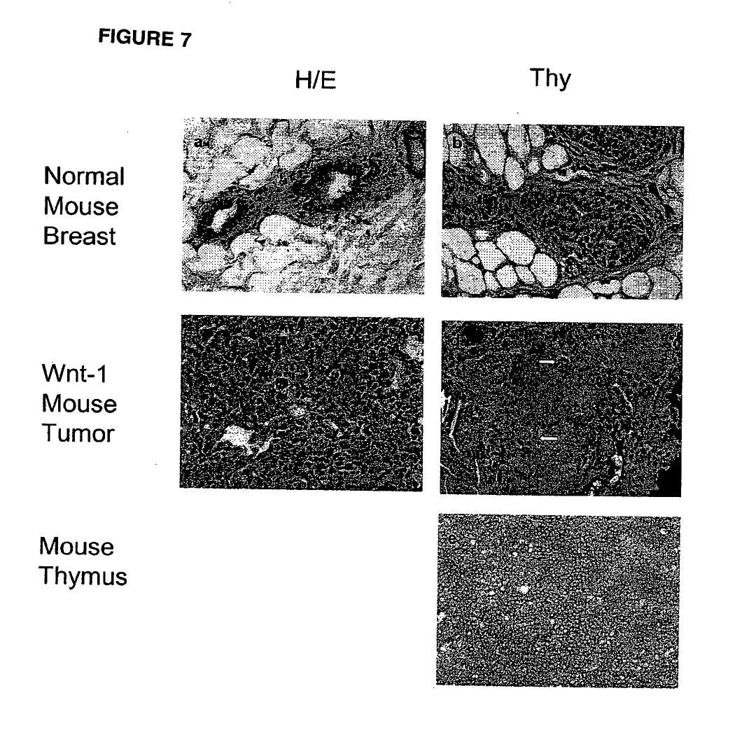

Immunohistochemistry was performed on normal mouse breast tissue and breast tumors in MMTV-Wnt-1 transgenic mice to localize cells that expressed Thy-1 within the tumor. Our CD24 antibody was not sensitive enough for these studies. Hematoxylin and Eosin (H & E) stains showed that the breast tumors were poorly differentiated invasive ductal carcinoma (FIG. 7C). In normal breast tissue, cells expressing Thy-1, which is thought to mark myoepithelial cells20 were located in the basal layer of ducts by immunohistochemistry (FIG. 7B). In tumors, Thy-1 was expressed by an occasional tumor cell within a nest of neoplastic cells (FIG. 7D). The staining pattern is consistent with the idea that tumorigenic cells with stem cell properties represent only a minority population within the tumor to give rise to the remaining bulk of cells that comprise the tumor.

A human Tumorigenic Breast Cancer Gene Expression Signature Identifies Tumorigenic Mouse Breast Cancer Cells

The relationship between the human and murine tumorigenic cancer cells was also studied. A human tumorigenic cancer cell gene signature was used to analyze the genes expressed by tumorigenic and non-tumorigenic cancer cells from MMTV-Wnt-1 mouse tumors. The human tumorigenic gene signature was generated by using 30,000 CD44⁺CD24^{-/lo}Lineage⁻ tumorigenic cancer cells that were isolated directly from 6 tumors from patients with breast cancer as well as from normal breast epithelial cells isolated from 3 patients' reduction mammoplasty tissue.

cDNA from each sample was used to probe Affymetrix 133A and 133B chips to determine genes that are differentially expressed by cancer stem cells and normal breast epithelial cells. As a result, a cancer stem cell gene signature was derived comprising 186 genes whose selection was based on two fold threshold for increased or decreased expression with a t-test P value of 0.005 across all samples.

To determine whether this 186 gene signature can distinguish tumorigenic cells from their non-tumorigenic progeny in breast tumors arising in MMTV-Wnt-1 mice, Thy1.1⁺CD24⁺CD45⁻ tumorigenic cancer cells and “Not Thy1.1⁺CD24⁺CD45⁻” cells from tumors in 3 different mice were used to make cDNA probe for the Affymetrix Mouse 430 2.0 oligonucleotide array. Of the 186 genes in the human signature, 160 are present on the mouse oligonucleotide chip. Remarkably, all of the mouse tumorigenic cells cluster with the human tumorigenic cancer cells while the non-tumorigenic cancer cells formed a distinct second group in a hierarchical cluster analysis (FIG. 5) suggesting that the mouse tumorigenic cells may be physiologically analogous to human tumorigenic cells.

The Human Tumorigenic Breast Cancer Gene Signature Predicts Death and Metastasis

Gene expression patterns of whole tumors can be used to predict survival and outcomes of patients with cancer. The 186 gene signature from human tumorigenic cancer cells, but not non-tumorigenic cancer cells, could predict survival in patients with breast cancer as well as cancer of other origins. Since the 186 gene signature identified a stem cell population in a solid tumor of another organism and could also be used to predict outcome in patients with breast cancer, it was reasoned that the 186 gene signature might predict the outcome of patients with other types of solid tumors.

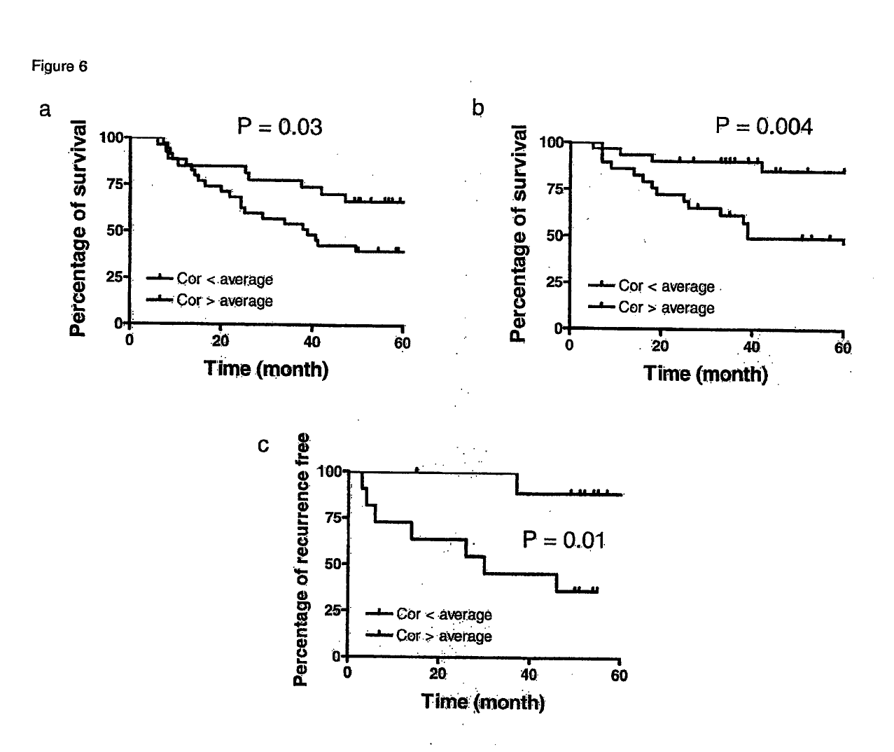

The ability of the 186 gene signature to predict metastasis and death in patients with lung cancer, meduloblastoma and prostate cancer was assessed (Bhattacharjee, A. et al., Proc Natl Acad Sci U S A 98:13790-5 (2001); Singh, D. et al., Cancer Cell 1:203-9 (2002)). Each microarray platform contained a subset of genes within each cancer stem cell gene signature (Supplemental Methods) the expression of which was then used to generate a cancer gene profile which was then compared to a cancer stem cell gene signature. Sixty-two lung cancer patients, 60 medulloblastoma patients, and 21 prostate cancer patients were divided into two groups based on their tumor's gene expression profile of our 186 tumorigenic gene signature. The predictive power of the cancer stem cell gene signature was then assessed by Kaplan-Meyer survival curves. Remarkably, this gene signature could identify patients destined to die of their disease in each of these populations (FIG. 6A-C). For all three cancer states, patients whose tumor gene expression profile highly correlated with the tumorigenic gene signature had less than 50% chance of survival at 60 months. Tumors whose gene expression profile showed poor correlation predicted high survival rates of >60% (lung cancer) to >80% (medulloblastoma and prostate cancer). The survival difference was found to be statistically significant (P=0.01-0.004).

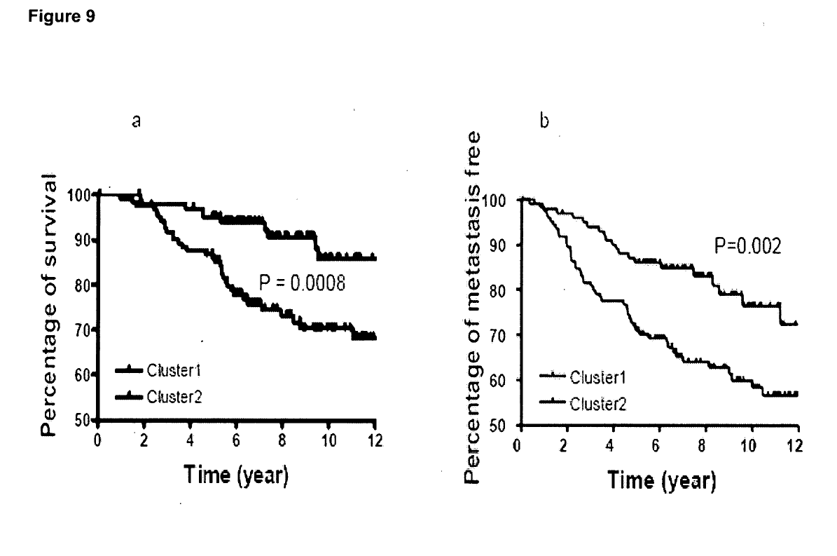

The ability of a mouse breast cancer signature derived from comparing TG and NTG cells to predict outcomes of human breast cancer was also determined. We identified human orthologs for 79 of the 121 TG/NTG genes, and 59 of these were present in previously published whole tumor gene expression data from 295 patients with stage I or II primary breast carcinoma (van de Vijver, M.J. et al., (2002) New Engl. J. Med. 347, 1999-2009). Hierarchically clustering the patients using only these 59 genes separated them into two groups with markedly different outcomes. Kaplan-Meier analysis revealed survival of 86% verses 59% and distant-metastasis free survival of 73% verses 54% at 12 years for the two groups (FIG. 9). Thus, genes differentially expressed in TG and NTG cells of MMTV-Wnt-1 mouse tumors predict clinical outcomes in patients with breast cancer.

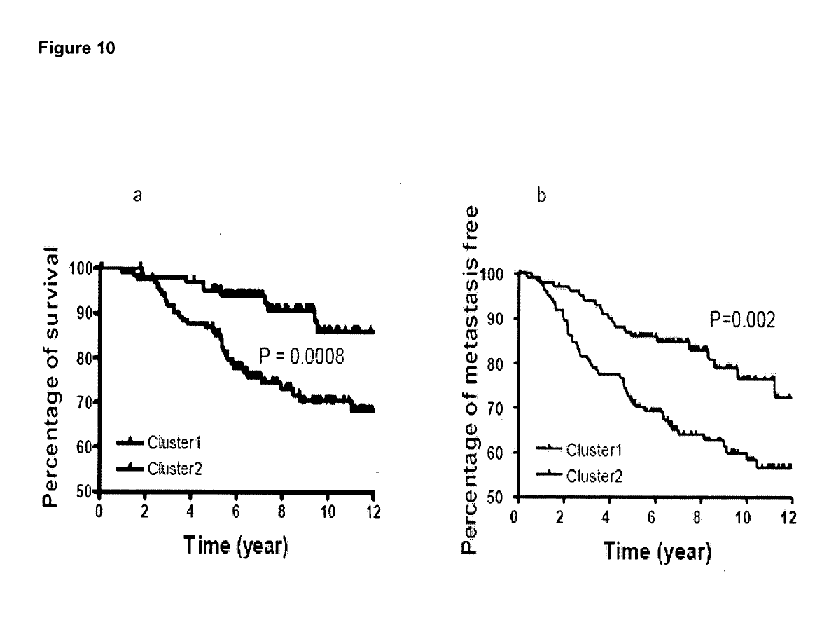

As the MMTV-Wnt-1 tumors are classified as estrogen receptor (ER) positive (Zhang, X. et al., (2005) Oncogene 24, 4220-31), and human ER+tumors have better prognosis than ER- tumors, we examined the distribution of ER+ and ER− tumors within our two prognostic groups. As expected, the vast majority of the ER− patients fell within the worse prognosis group, as did patients with other poor prognostic markers such as basal cell subtype and poor histologic grade. However, the predictive power of the TG/NTG signature was not simply a result of identifying ER+ tumors, since the signature also subdivided the ER+patients alone into good and poor prognostic groups (FIG. 10). Thus, the outcomes predicted by the TG/NTG gene signature are not simply a recapitulation of estrogen receptor status.

These results demonstrate for the first time that tumorigenic and non-tumorigenic cells can be prospectively identified in vivo in a murine solid tumor. In MMTV Wnt-1 transgenic mice, a minority population of phenotypically distinct cancer cells defined by the cell surface marker expression Thy1⁺CD24⁺CD49f⁺CD45⁻ is highly enriched for cells able to form tumors when transplanted into syngeneic mice recipients. In multiple serial passages, the tumorigenic cancer cells are able to give rise to additional Thy1⁺CD24⁺CD49f⁺CD45⁻ tumorigenic cells as well as larger numbers of aberrantly differentiated progeny that express a mature epithelial marker, CK19. This fulfills the criteria that define stem cells; that is the ability to self-renew, to differentiate and to proliferate extensively.

That a transformed normal stem or progenitor cell may be the tumorigenic cancer cell while some of their non-tumorigenic progeny represent aberrantly differentiated cells has been demonstrated in acute leukemia (Bonnet, D. and Dick, J. (1997); Jamieson, C.

H. et al. (2004)), however, this data provides the first in vivo evidence suggesting that this concept might also be true for some epithelial tumors. Thus far, cancer stem cells have been demonstrated to exist in only certain solid human cancers namely breast cancer and brain cancers. In both these cases, only a minority population of cancer cells has the exclusive ability to form tumors in immunodeficient mice (Al-Hajj, M et al. (2003); Singh, G. (2004)). Although the behavior of this subpopulation of tumorigenic cells is similar to stem cells by their ability to self-renew and reconstitute the heterogeneity of the original tumor, the cellular origin of these cancer stem cells in solid tumors has remained elusive. Normal stem cells are an obvious target of transformation given their similarity to cancer stem cells in their ability to self-renew and proliferate. Supporting this, CD133 marks both normal and neoplastic brain cancer stem cells (Singh, S. K. et al. Nature 432:396-401 (2004); Uchida, N. et al., Proc Natl Acad Sciences USA 97:14720-5 (2000); Clarke, M. F. Nature 432: 281-2 (2004)).

This model is further bolstered by a recent study that identified a population of murine lung epithelial cells with stem cell properties in culture that were expanded in early lung tumors (Kim, C. F. et al., Cell 121:823-35 (2005)). However, differences in the tumor forming ability of different populations of cancer cells in this study was not determined.

The MMTV-Wnt-1 mouse model of breast cancer has permitted investigation into the relationship of tumorigenic cancer cells with their normal counterparts. MMTV-Wnt-1 tumors were shown to contain both luminal epithelial and myoepithelial cells and in certain cases, harbored a common second mutation, suggesting that these tumors arose from a common progenitor (Li, Y. et al., Proc Natl Acad Sci USA 100:15853-8 (2003)).

These studies, however, did not determine whether the heterogeneous populations differed in their ability to form tumors. An alternative explanation is that environmental influences caused variations in phenotype and all of the populations of cancer cells could form new tumors (Reya, T. et al., (2001)). These results demonstrate that minority populations of Thy1⁺CD24⁺CD49f⁺CD45⁻ cells in normal murine mammary gland glands and MMTV-Wnt-1 tumors have the ability to generate new mammary ducts and tumors, respectively. Furthermore, the non-tumorigenic cancer cells preferentially express cytokeratin 19, which is thought to be an epithelial cell differentiation marker (Gudjonsson, T. et al. Genes Dev. 16:693-706 (2002); Blyszczuk, P. et al. Int J Dev Biol 48:1095-104 (2004)).

This suggests that the non-tumorigenic cells have undergone abnormal differentiation concurrent with a loss in the ability to self-renew. Thus, in this model system, evidence that a cell early in the hierarchy of mammary growth and development is likely the target of transformation into a tumor-regenerating cell is provided.

Gene signatures to predict survival in a particular cancer have been derived from both computer analysis of expression data and the identification of genes expressed in response to cellular processes such as wound repair (Chang; H. Y. et al., Proc Natl. Acad. Sci. USA 102:3738-43 (2005)). That a gene signature derived from human tumorigenic breast cancer cells can predict survival and metastasis in multiple types of human solid tumors as well as distinguish tumorigenic from nontumorigenic cancer cells in a murine cancer model suggests that this signature represents some fundamental property of poor prognosis cancers. Since this pattern can be used to distinguish tumorigenic from non-tumorigenic mouse cancer cells, a simple explanation is that identity with the signature derived from these 6 patients' tumorigenic cancer cells represents cancer stem cell frequency in a particular tumor. However, alternative explanations are equally feasible. For example, this signature could reflect fundamental properties of the tumorigenic cells from these patients that confer poor prognosis such as proliferative capacity, invasiveness, inherent resistance to therapy or the maturation level of the cancer cells in a particular tumor sample that is shared with the murine tumorigenic cancer cells. It has long been recognized by clinical pathologists that poorly differentiated tumors are considered more aggressive with poorer prognosis. Since many oncogenic mutations block differentiation, it follows that mutations that inhibit phenotypic maturation would give rise to tumors whose gene signature shares similarities with the 186 gene signature.

Only after genomic analysis of hundreds, maybe thousands, of patients' tumorigenic cancer cells will these models be distinguished. The tumorigenic cancer cell signature was derived from 6 patients, and it is certainly possible that cancer stem cells from different tumors will have unique gene signatures of their own. Such a large analysis is unlikely to be completed in the immediate future due to technical difficulties. Because of the paucity of tumorigenic cells in tumors, it is not possible to obtain enough cells to make probe for microarrays from most primary breast tumors. Nonetheless, this 186 gene signature clearly has demonstrated its prognostic power in certain human cancers.

Although transgenic mouse models may be considered artificial systems and may at times differ in pathology from human breast cancers, they nonetheless are valuable tools that will enable us to meet the challenge of dissecting molecular pathways involved in both normal and cancer stem cell biology. The ability of the human tumorigenic cancer cell signature to identify tumorigenic and non-tumorigenic cell populations suggests that the MMTV-Wnt-1 breast tumors will be a useful tool to model the biology of human tumorigenic cancer cells.

Experimental Procedures:

Tumor Harvest and Dissociation:

MMTV-Wnt-1 FVB/NJ (Jackson Laboratory 002934) breeding colonies were established by crossing male transgenic mice with wild-type female FVB/NJ (Jackson Laboratory 001800) mice. Resulting female transgenic mice were separated and allowed to grow de novo breast tumors. Tumors were harvested when the tumors were approximately 1-2 cm³ (2-2.5 grams). Medium 199 (Gibco BRL) with 20 mM Hepes buffer was used to wash the tumor. After washing, the tumor was minced with a razor blade in 5 ml of medium 199 with 20 mM of Hepes buffer. The minced tumor was suspended into 20 ml of medium 199 with 20 mM of Hepes buffer. 100 Kunitz U of DNAse I (Sigma D4263) was then added. Collagenase digestion was accomplished with Liberase Blendzyme 2 (Roche 1998433) 8 Wunsch U and Liberase Blendzyme 4 (Roche 1988476) 8 Wunsch U. Digestions lasted for a total of approximately 2.5 hours at 38° C. Every 30 minutes, dissociation was aided by manually pipetting several times through a sterile 10 ml pipette. After two hours of digestion, another 100 Kunitz U of DNAse I was added. Once digested, 80 ml of RPMI (BioWhittaker) with 10% calf serum (CS) (HyClone) was added to the digestion solution to inactivate the collagenases. Nylon 40 μm filters were used to filter the sample. Cells were centrifuged at 190 RCF for 5 minutes. The cell pellet was resuspended in 5 ml of ACK buffer for red blood cell lysis for one minute. HBSS (BioWhittaker) with 2% heat-inactivated calf serum (HICS) was used to dilute the ACK buffer and cells filtered again through a nylon 40 μm filter. The filtered cells were spun down and resuspended in HBSS with 2% HICS (staining media).

Cell Staining and Flow Cytometry:

The single cell suspension was counted on a hemacytometer. Cells were stained at a concentration of lx 106 cells per 100 μL of HBSS with 2% HICS in a total volume of 1 ml. 10 μL of rabbit IgG (1 mg/ml) was then added. Antibodies were then added at appropriate dilutions (CD24-PE, eBioscience; Thy1.1-APC, eBioscience; CD49f-FITC, BD Pharmingen; CD45-PE-Cy5, BD Pharmingen). Appropriate controls for calibration of the flow cytometer were also prepared. Staining duration was for 20 minutes on ice with light agitation of the staining vessels every 5 minutes. Cells were then washed with HBSS with 2% HICS and resuspended in HBSS with 2% HICS containing 7-aminoactinomycin D (7-AAD, 1 μg/ml final concentration) or 4′-6-Diamidino-2-phenylindole (DAPI, 1 μg/ml final concentration).

The stained specimens were then analyzed using FACSVantage (BD Bioscience) or FACSAria with either Diva or CellQuest software (BD Bioscience). Different populations of cells were isolated by setting selection criteria. Side scatter and forward scatter profiles were used to reduce doublets. Viable cells were selected for by eliminating 7-AAD or DAPI positive cells. Hematopoietic cells were eliminated by gating out all CD45 positive cells. Cells with appropriate CD24, Thy1.1, and CD49f status were then collected. To reduce the rate of contamination of cells that do not fit the requested cell profile, all collected cells were sorted a second time (double sort). A small sample of the double-sorted cells were reanalyzed for purity. Final cell purity was greater than 95%. The cell counter of the flow cytometers was used to determine cell numbers. Cells were collected into RPMI or HBSS with 2% HICS.

Tumor Injection:

FVB/NJ female mice (4-8 weeks of age) were injected intra-peritoneally with 200 ul of a mixture of ketamine (12 mg/ml) (Fort Dodge) and xylazine (0.8 mg/ml) (Lloyd) in PBS. Sorted cells were suspended in 100 μl of collection media which was then mixed with 100 μl of Matrigel (BD Biosciences 354234). The cell mixture was then injected near the upper mammary fat pads of the mice using a 23 gauge 1 inch needle which tracked caudally subcutaneously from the anterior rib border. Vetbond (3M 1469SB) was used to close the injection site. Mice were observed weekly for 6-8 months for tumor formation. Some resultant tumors were analyzed and injected in the same manner as de novo tumors.

Mammary Gland Harvest and Dissociation:

All mammary glands from donor female 4-6 week old C57BL/Kα-1.1/Thy1.1 mice were harvested by surgical resection and immediately placed in 9 ml ice cold Media 199 buffered with 25 mM HEPES and antibiotic/antimycotic solution (penicillin, streptomycin, actinomycin) (PSA). Glands were roughly minced in media using a sterile razor blade. 1 ml 2000 unit/ml sterile filtered collagenase type III (Worthington Mannheim) was then added and tissue suspension was incubated at 37° C./5% CO2 for 1.5-2 hours, with mechanical aspiration of tissue suspension performed every 15-20 min. At end of digestion, 10 ml of DMEM with 10% calf serum and PSA was added to inactivate the collagenase. The dissociated cell suspension was then filtered through a 40 μM nylon mesh filter to obtain a single cell suspension.

Mammary Cell Staining and Flow Cytometry:

The dissociated cell suspension was then counted by hemacytometer and centrifuged at 150 RCF for 5 min. The cell pellet was resuspended at a concentration of 1×108 cells/ml with HBSS with 2% HICS (staining media). Cells were stained at a concentration of 10×10⁶ cells per 100 μL of staining media. 1 μL of rat IgG (1 mg/ml) was added per 100 μL of staining volume to block nonspecific binding. Cells were stained with CD24-PE, Thy1.1-APC, CD49f-FITC, CD45-PE-Cy5, and biotinylated Thy1.2 (BD Pharmingen) for 15 minutes on ice and washed twice with staining media. Secondary streptavidin-PE-Cy7 (BD Pharmingen) was used to detect staining by biotinylated Thy1.2. Cells were then washed and resuspended in staining media containing 4′-6-Diamidino-2-phenylindole (DAPI, 1 μg/ml final concentration) at 1:1000 dilution. The stained specimens were then analyzed using FACSVantage with either Diva or CellQuest software as described above for tumor samples. Cells with appropriate CD24, Thy1.1, and CD49f status were double sorted for and collected into Ham's F-12 (Gibco BRL) supplemented with 1% FBS (HyClone), PSA, Insulin-Selenium-Transferrin (Gibco BRL), cholera toxin (Sigma), heparin (Sigma), B-27 supplement (Gibco BRL), Glutamax (Gibco BRL), EGF (Gibco BRL), and 25 mM HEPES (Gibco BRL).

Mammary Cell Engraftment:

Recipient C57BL/6J female mice (3-4 weeks of age) were anesthetized using aforementioned protocol at 1 μL/Gm body weight. Sorted cells from 4-6 week old C57BL/Kα-1.1/Thy1.1 mice (donor) were mixed in a 1:1 ratio with Matrigel and injected into surgically cleaned fourth mammary fat pads of recipient mice . Injections were made into the cleared fat-pad near the milk bud (<1 mm) in a total volume of 10 μL with a sterile Hamilton syringe. Recipient mice were closed using sterile autoclips (myneuron.com) and Vetbond (3M 1469SB). Engrafted mice were placed under radiant heat lamp until recovery and then given antibiotic water (ciprofloxacin, 16 μg/ml) for 14 days. Autoclips were removed after 14 days. At 6 weeks after initial engraftment, recipient mice were sacrificed and cleared fat pad regions were analyzed by visual observance for ductal outgrowth as well as flow cytometric analysis for Thy1.1, Thy1.2, CD24, CD49f, and CD45 according to above-mentioned protocol.

Mouse Array Analysis:

Three MMTV-Wnt-1 tumors were harvested and cells sorted into CD24⁺Thy⁺CD45− and “Not CD24⁺Thy⁺CD45−” populations of 10,000 cells each using the above mentioned protocol. RNA isolation was accomplished using RNAqueous-Micro (Ambion #1931). The RNA was used by the University of Michigan to produce array data using NuGen Ovation Biotin labeling system and Affymetrics Mouse 430 2.0 GeneChips.

Real-Time PCR:

Triplicate collections of 1000 cells of both CD24⁺Thy⁺CD45− and “Not CD24⁺Thy⁺CD45−” cells were collected in Trizol. RNA was collected and cDNA made by common molecular biology techniques. TaqMan Gene Expression Assays (Applied Biosystems) was utilized in performing RT-PCR. Hprt-1 (Applied Biosystems Mm0046968) and Krt1-19 (Applied Biosystems Mm00492980) detection oligonucleotides were obtained and RT-PCR assays done per product protocol in 20 uL PCR volumes. RT-PCR reactions were run in the University of Michigan Array Core.

Supplemental Methods:

Statistical Analysis of Array Data:

Array annotation: For Affymetrix arrays, the latest annotation files were downloaded from the Affymetrix web site (can be found on the world wide web at URL: affymetrix.com) and used for all further analysis. For clone-based arrays, the annotation in the downloaded data file was directly used. For the Rosetta/NKI oligonucleotide array, oligonucleotide sequences were downloaded from the Rosetta website and a Blast was performed between oligonucleotides and sequences from NCBI Genes database to annotate the array. Array elements from different arrays are mapped to each other by gene symbols.

Data transformation: For Affymetrix scoring of the downloaded dataset, the signal intensity values of probes were transformed into log ratios using the average intensity of the probe across all samples within dataset as denominator. Probes with an average signal intensity value across all samples within the dataset smaller than 20 were filtered out. If the signal intensity value was less than 20, then it was converted to 20. For the three datasets (early lung cancer, Medulloblastoma and Prostate cancer), if the signal intensity value was larger than 16000, then it was converted to 16000. This is the same convention as described by Ramaswamy and colleagues (Ramaswamy, S., et al., Nat Genet 33:49-54 (2003)).

Cluster analysis: Average linkage clustering was carried out using the Cluster software and visualized using TreeView software (Eisen et al. 1998; sofware can be viewed on the world wide web at URL: bonsai.ims.u-tokyo.acjp/-mdehoon/software/cluster/).

Mouse/Human probe set mapping for genes in gene signature: An ortholog mapping file (on the world wide web at URL: Mouse430_{—}2_ortholog.csv) between human probe sets and mouse probe sets was downloaded from Affymetrix web site. If there were multiple mouse probe sets mapped to one human probe set, the one with the highest signal intensity across 6 samples was used for further analysis. 160 out of 186 genes in the gene signature were mapped to mouse orthologs.

Survival analysis: The Pearson correlation coefficient between each patient and gene signature was used as a prognostic value for the outcome of the patient. The Kaplan-Meier survival analysis was performed using the GraphPad Prism version 4.03 software (GraphPad Software Inc, San Diego, Calif., USA). Statistical significance of the difference between the curves from different groups of patients was assessed using log-rank tests. An artificial threshold of 0 or average correlation value for the correlation was chosen to separate patients into different groups. Univariate and multivariate survival analysis by Cox proportional hazard method was carried out by using the software package R 2.1.0 (www.r-project.org). Overall survival was defined by death from any cause.

Immunohistochemistry:

Immunohistochemistry was performed on 4 uM paraffin-embedded tumor sections with a Dako Autostainer Universal Staining System. Staining with a 1/100 dilution of anii-Thy-1 (eBioscience, 1:400, clone G7) was performed using a Mouse on Mouse Animal Research Kit (Dako). All sections were processed for antigen retrieval by boiling under pressure in sodium citrate buffer (lOmM sodium citrate, 0.05% Tween 20, pH 6.0) for 10 minutes and allowed to cool. Thy-l sections were blocked with 3% H₂0₂ for 10 minutes, followed by biotin blocking system (Dako).

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way. All publications, patents and patent applications cited herein are incorporated by reference in their entirety into the disclosure.

What is claimed is:

1. An isolated population of solid tumor stem cells, the population comprising at least 75% solid tumor stem cells and less than 25% solid tumor cells, wherein the solid tumor stem cells:
(a) are tumorigenic;
(b) express Thy1, CD24, CD49f; and
(c) do not express detectable levels of CD45;
and wherein the solid tumor cells are non-tumorigenic.

2. The isolated population of claim 1, wherein the solid tumor stem cells are breast cancer stem cells.

3. The isolated population of claim 1, wherein one or more of the solid tumor stem cells contain a polynucleotide vector.

4. The isolated population of claim 3, wherein the polynucleotide vector is a viral vector or a plasmid.

5. The isolated population of claim 3, wherein the polynucleotide vector contains a reporter polynucleotide.

6. The isolated population of claim 5, wherein the reporter polynucleotide provides a detectable signal when active in a solid tumor stem cell.

7. The isolated population of claim 1, wherein one or more of the solid tumor stem cells further comprise a recombinant polynucleotide.

8. The isolated population of claim 7, wherein the recombinant polynucleotide is integrated into a chromosome of the solid tumor stem cell.

9. The isolated population of claim 1, wherein the solid tumor stem cells are capable of forming a new tumor upon transplantation into a host animal.

10. The isolated population of claim 9, wherein the host animal is an immunocompromised mouse.

11. The isolated population of claim 1, which is situated in a culture medium.

12. The isolated population of claim 1, wherein the solid tumor stem cells are affixed to a substrate.

13. The isolated population of claim 1, wherein the solid tumor stem cells have been treated to reduce proliferation.

14. The isolated population of claim 1, wherein the solid tumor stem cells have been treated to increase proliferation.

15. An enriched population of solid tumor stem cells, the population comprising solid tumor stem cells and solid tumor cells, wherein the solid tumor stem cells:
(a) are enriched at least two-fold;
(b) are tumorigenic;
(c) express Thy1, CD24, CD49f; and
(d) do not express detectable levels of CD45.

16. The enriched population of solid tumor stem cells of claim 15, wherein the population is enriched at least 5-fold.

17. A method of enriching for a population of solid tumor stem cells, the method comprising:
(a) dissociating a murine solid tumor to obtain dissociated cells;
(b) contacting the dissociated cells with a first reagent that binds Thy1, a second reagent that binds CD24, a third reagent that binds CD49f, and a fourth reagent that binds CD45; and
(c) selecting solid tumor stem cells that bind the first, second and third reagents and do not bind to the fourth reagent.

18. The method of claim 17, further comprising isolating the selected solid tumor stem cells.

19. The method of claim 17, wherein the first, second, third or fourth reagent is an antibody.

20. The method of claim 17, wherein the first, second, third or fourth reagent is conjugated to a fluourochrome or magnetic particle.

21. The method of claim 17, wherein the selection of solid tumor stem cells is performed by flow cytometry, fluorescence activated cell sorting, panning, affinity column separation or magnetic separation.

22. The method of claim 17, wherein the murine solid tumor stem cells are breast cancer stem cells.

23. The method of claim 17, wherein the dissociated cells are contacted with the first, second, third, and fourth reagents concurrently.

24. The method of claim 17, further comprising: (d) introducing at least one selected cell to a culture medium that supports growth of tumor stem cells; and (e) proliferating the selected cell in the culture medium.

25. The method of claim 24, further comprising: (f) contacting the proliferated cell with a test compound; and (g) determining the effect of the test compound on the proliferated cell.

26. A method for analyzing an enriched population of solid tumor stem cells for a gene expression pattern, the method comprising:
(a) obtaining an enriched population of solid tumor stem cells, wherein
(i) the solid tumor stem cells are derived from a solid tumor;
(ii) the solid tumor stem cells expresses the cell surface markers Thy1, CD24, and CD49f;
(iii) the solid tumor stem cells do not express CD45;
(iv) the solid tumor stem cells are tumorigenic; and
(v) the solid tumor stem cell population is enriched at least 2-fold relative to unfractionated tumor cells; and
(b) analyzing the enriched population for a gene expression pattern.

27. The method of claim 26, wherein the analysis is by a method selected from the group consisting of sequencing, high throughput screening, use of a microarray, use of analytical software for data collection and storage, use of analytical software for flexible formatting of data output, use of analytical software for statistical analysis of individual spot intensities to provide grouping and cluster analyses, and use of analytical software for linkage to external databases.

28. A method for analyzing an enriched population of solid tumor stem cells for protein expression patterns, the method comprising:
(a) obtaining an enriched population of solid tumor stem cells, wherein
(i) the solid tumor stem cells are derived from a solid tumor;
(ii) the solid tumor stem cells express the cell surface markers Thy1, CD24, and CD49f;
(iii) the solid tumor stem cells do not express CD45;
(iv) the solid tumor stem cells are tumorigenic; and
(v) the enriched population is enriched at least 2-fold for solid tumor stem cells relative to unfractionated tumor cells; and
(b) analyzing the enriched population for a protein expression pattern.

29. The method of claim 28, wherein the analysis is by a method selected from the group consisting of mass spectrometry, high throughput screening, use of a microarray, use of analytical software for data collection and storage, use of analytical software for flexible formatting of data output, use of analytical software for statistical analysis of individual spot intensities to provide grouping and cluster analyses, and use of analytical software for linkage to external databases.

30. A method for determining an effect of a test compound on a solid tumor stem cell, the method comprising:
(a) obtaining a solid tumor stem cell, wherein;
(i) the solid tumor stem cell is derived from a solid tumor;
(ii) the solid tumor stem cell expresses the cell surface markers Thy1, CD24 and CD49f;
(iii) the solid tumor stem cell does not express CD45; and
(iv) the solid tumor stem cell is tumorigenic;
(b) contacting the solid tumor stem cell with the test compound; and
(c) determining the response of the solid tumor stem cell to the test compound.

31. The method of claim 30, wherein the solid tumor stem cell is a breast cancer cell.

32. The method of claim 30, wherein the solid tumor stem cell is localized in a manner selected from the group consisting of: in a monolayer in culture, in suspension in culture, and affixed to a solid surface.

33. The method of claim 30, wherein the contacting is effected at more than one concentration of the test compound being tested.

34. The method of claim 30, wherein the contacting is effected using a microfluidic method.

35. The method of claim 30, wherein the determination of the response of the contacted cell to the test compound comprises assaying for an effect selected from the group consisting of tumor formation, tumor growth, tumor stem cell proliferation, tumor cell survival, tumor cell cycle status, and tumor stem cell survival.

36. The method of claim 35, wherein the test compound is attached to a solid surface.

37. The method of claim 36, wherein the test compound is attached to a solid surface as a microarray.

38. The method of claim 30, wherein the test compound is in a set of other molecules.

39. The method of claim 30, wherein the test compound is in an array of other molecules.

40. The method of claim 30, further comprising (d) identifying the target in the contacted cell with which the test compound interacts.

41. A method for determining an effect of a test compound on a solid tumor stem cell, the method comprising:
(a) obtaining a solid tumor stem cell, wherein;
(i) the solid tumor stem cell is derived from a solid tumor;
(ii) the solid tumor stem cell expresses the cell surface markers Thy1, CD24 and CD49f;
(iii) the solid tumor stem cell does not express CD45; and
(iv) the solid tumor stem cell is tumorigenic;
(b) transplanting the obtained cell into an immunocompromised mouse;
(c) administering a test compound to the immunocompromised mouse; and
(d) determining the response of the transplanted solid tumor stem cell to the test compound.

42. The method of claim 41, wherein the solid tumor stem cell is a breast cancer cell.

43. The method of claim 41, wherein the enriched population of solid tumor stem cells is an isolated solid tumor stem cell.

44. A method for producing a genetically modified solid tumor stem cell, the method comprising:
(a) obtaining a solid tumor stem cell, wherein;
(i) the solid tumor stem cell is derived from a solid tumor;
(ii) the solid tumor stem cell expresses the cell surface markers Thy1, CD24 and CD49f;
(iii) the solid tumor stem cell does not express CD45; and
(iv) the solid tumor stem cell is tumorigenic; and
(b) genetically modifying the obtained solid tumor stem cell.

45. The method of claim 44, wherein the solid tumor stem cell is a breast cancer cell.

46. The method of claim 44, wherein the genetic modification is performed in vitro.

47. The method of claim 44, wherein the genetic modification is performed in vivo.

48. The method of claim 44, wherein the genetic modification is introduction of a plasmid into the solid tumor stem cell.

49. The method of claim 44, wherein the genetic modification is introduction of a viral vector into the solid tumor stem cell.

50. The method of claim 49, wherein the viral vector has been modified to express a protein that recognizes an antigen on the solid tumor stem cell.

51. The method of claim 49, further comprising: (c) examining the effect of the genetic modification on tumor formation, tumor growth, tumor cell proliferation, tumor cell survival, tumor stem cell survival, tumor stem cell proliferation, tumor cell cycle status, or tumor stem cell frequency.

52. A method for determining a prognosis of a cancer patient, the method comprising:
(a) obtaining a cell sample from the patient:
(b) determining a gene signature pattern of the cell sample;
(c) comparing the gene signature pattern of the cell sample to the gene signature pattern of an analogous murine tumor; and
(d) evaluating whether the gene signature pattern of the cancer patient is similar to the gene signature pattern of the murine tumor.

53. The method of claim 52, wherein a positive prognosis results from a difference in the gene signature patterns.

54. The method of claim 52, wherein a negative prognosis results from similar gene signature patterns.

55. The method of claim 52, wherein (b) further comprises isolating solid tumor stem cells from the cell sample.

56. The method of claim 52, wherein (b) further comprises array amplification of gene signature targets.

57. The method of claim 52, wherein determining the expression levels comprising the gene signature is by measuring the expression of a corresponding polypeptide.

58. The method of claim 57, wherein the polypeptide is detected by immunohistochemical analysis on the cell sample using an antibody or antigen binding fragment that binds the polypeptide.

59. The method of claim 57, wherein the polypeptide is detected by ELISA assay using an antibody or antigen binding fragment that binds the polypeptide.

60. The method of claim 57, wherein the polypeptide is detected using an antibody array comprising an antibody or antigen binding fragment that binds the polypeptide.

61. The method of claim 52, wherein the gene signature comprises genes in Table 1.

62. A method of identifying the presence of solid tumor stem cells in a subject suspected of having cancer, wherein the method comprises:
(a) obtaining a biological sample from the subject;
(b) dissociating cells of the sample;
(c) contacting the dissociated cells with a reagent selected from the group consisting of: a first reagent that binds Thy1, a second reagent that binds CD24; a third reagent that binds CD49f, and a fourth reagent that binds CD45; and
(d) detecting solid tumor stem cells that bind at least the first, second or third reagent, and do not bind to the fourth reagent.

63. The method of claim 62, wherein the solid tumor stem cells are breast cancer stem cells.

64. The method of claim 62, wherein the first, second, third, or fourth reagent is an antibody.

65. The method of claim 62, wherein the first, second, third, or fourth reagent is conjugated to a fluorochrome or magnetic particle.

66. The method of claim 62, wherein the detection step is performed by flow cytometry, fluorescence activated cell sorting, panning, affinity column separation, or magnetic selection.

67. The method of claim 62, wherein the biological sample is from a primary tumor.

68. The method of claim 62, wherein the method further comprises selecting a treatment course of action for the subject.

69. The method of claim 68, wherein the treatment comprises administration of a Notch 4 pathway inhibitor to the subject.

70. The method of claim 68, wherein the treatment comprises administration of an antibody to the subject.
